(19) United States
(12) Patent Application Publication
(10) Pub. No.: US 2016/0051176 A1
(43) Pub. Date: Feb. 25, 2016

(54) Urine Monitoring Systems and Methods

(71) Applicant: C.R. Bard, Inc., Murray Hill, NJ (US)

(72) Inventors: Ruben Ramos, Loganville, GA (US); Michael O'Grady, Monroe, GA (US); Fungbor Chen, Covington, GA (US)

(21) Appl. No.: 14/764,948

(22) PCT Filed: Mar. 17, 2014
(86) PCT No.: PCT/US14/30833

§ 371 Date: Jul. 30, 2015

Related U.S. Application Data
(60) Provisional application No. 61/794,917, filed on Mar. 15, 2013.

(51) Int. Cl. A61B 5/20; A61B 10/00; A61B 5/00

(52) U.S. Cl. CPC A61B 5/208; A61B 5/0004; A61B 10/007; A61B 5/7278; A61B 2562/0214

(57) ABSTRACT

Fluid/urine monitoring devices and/or systems are provided for monitoring fluid output, including volume and flow rate. One high resolution, low cost electronic urine monitoring device and system collects urine and includes a capacitance sensor. The capacitance of the capacitance sensor may be correlated with fluid content and be used to identify urine volume and flow rate. Another high resolution, low cost flow meter is placed in line with drainage tubing and uses a capacitance sensor to measure fluid output without collecting the fluid. Other low cost urine monitoring devices use pressure based or weight based measurement sensors to measure volume and flow rate.

### PRIORITY

This application claims the benefit of priority to U.S. Provisional Application No. 61/794,917, filed Mar. 15, 2013, which is incorporated by reference in its entirety into this application.

### BACKGROUND

Urinary drainage containers or bags are conventionally used in hospitals and health care facilities when it is necessary to collect urine from a catheterized patient over a period of time. These containers/bags permit the patient to remain in bed, without having to be moved to use a bathroom or a bedpan. Urinary drainage systems may include a catheter (e.g., a Foley catheter), a collection container/bag (e.g., a bag made of a polymeric material or PVC film), and tubing connecting the Foley catheter to the collection container/bag. In operation, the patient is first catheterized, and the catheter is then connected to the drainage container/bag through a length of tubing. The urine drains through the catheter, the tubing, and then finally into the collection container/bag. The urine may be moved from the catheter into the collection bag solely due to gravitational forces. On average, about 80-90 mL of urine are produced in 1 hour.

It can be important for patient care to track the patient's urine flow rate and the volume of urine produced by the patient. Irregularities in urine flow rate or volume can signal to the clinician that the patient is suffering certain problems. In some instances, urine volume is tracked by removing urine collection containers/bags after they are filled and then measuring the volume post-collection, but this fails to track volume and flow rate during urination and can delay detection of problems. Certain automated urine output sensing devices rely on an ultrasound pulsed echo sensor to detect fluid levels and calculate urine flow. However, pulsed echo ultrasonic measurements suffer from certain limitations, including that they are relatively expensive and have accuracies limited by meter angle.

Another potential problem with urine drainage systems is that urine may columnate within the drain lumen of the catheter and/or other tubing instead of continuously flowing when the urine level reaches the drainage holes. Surface tension of the catheter material, e.g. silicone, may cause or contribute to the columnation and prevent continuous flow. When this columnation occurs it is difficult, if not impossible, to get an accurate measurement of flow rate. For example, the initial flow of urine can be delayed by the columnation and thereby prevent accurate measurement of initial flow. Additionally, columnation can result in a bolus of fluid forming before surface tension is overcome. When the resulting bolus amount of fluid is released, it may cause error in the measurements and may be above the capacity of an attached flow meter. Another potential drawback is that columniation can leave residual fluid “backed-up” in bladder and leave residual fluid in the drain lumen, which can lead to sanitation and health issues.

This disclosure relates to low cost, high resolution fluid monitoring devices and systems for monitoring/measuring fluid volume, flow rate, and other parameters. The devices and systems disclosed may be used as urine monitoring devices/systems, or may be used to monitor other fluids in various applications. Additionally, the disclosure relates to ways of improving fluid flow through the system, thereby improving measurements and helping to prevent unwanted fluid from remaining in the system.

### SUMMARY

Described herein are fluid/urine monitoring devices and systems including features believed to provide advantages over existing fluid/urine meters. The reliable, low cost fluid (e.g., urine) monitoring devices and/or systems of this disclosure include without limit capacitance-based measurement systems, pressure-based measurement systems, weight-based transducer measurement systems (e.g., load cell or strain gage systems), and/or other measurement systems.

In one embodiment, capacitance-based measurement principles are used to measure urine output. This embodiment provides a high resolution, low cost electronic volume and flow rate urine meter and recorder. This embodiment implements an autonomous inexpensive circuit that indicates volume and computes flow rate arbitrary of the size or shape of the bag/container. Some benefits of this embodiment include reducing the caregiver time spent, including by eliminating the need to record manually these critical parameters. Further, this embodiment helps to eliminate human error associated with reading the measurements. In order to measure the volume, flow rate, composition, etc. of urine, a sensitive probe with variable permittivity capacitance sensor is designed.

In one embodiment, a fluid monitoring system includes a container for collecting a fluid, and a capacitance sensor attached to the container and configured to act as a capacitor to sense a physical property of the fluid as it collects in the container. The fluid monitoring system also includes a microcontroller programmed to calculate a volume of the fluid based on data received from the capacitance sensor, e.g., a measurement of capacitance of the capacitance sensor, as the fluid collects in the container. The measurement/data of capacitance may be indirectly measured from the capacitance sensor using an oscillator, a CVD, a Bridge method, a Charge-Based method, and/or a CSM method. The microcontroller may include software programmed to transmit the volume with a unique identifier to distinguish the volume transmitted by the fluid monitoring system from data transmitted by other monitoring systems.

The capacitance sensor can have a generally coplanar electrode structure formed from only two parallel electrodes, or an interdigital electrode structure. The electrode structures may be formed from conductive ink on an external surface of the container.

The fluid monitoring system may also include a reference capacitor configured to measure a dielectric property of air and a compensation capacitor configured to measure a dielectric property of the fluid, the microcontroller programmed to continuously estimate a dielectric constant of the fluid based on data received from the reference capacitor and the compensation capacitor, and thereby facilitate automatic compensation for variations in the composition and/or conductivity of the fluid being measured. The fluid monitoring system may include a wireless transceiver or transmitter for transmitting the measurements, including volume and flow rate, to a separate device (e.g., a computer, monitor, smart phone, etc.).

In one embodiment, a method of measuring fluid volume includes providing a urine monitoring device that includes a container for collecting a fluid, a capacitance sensor attached to the container and configured to act as a capacitor to sense a physical property of the fluid, and a microcontroller programmed to use data from the capacitance sensor to calculate a volume and/or a flow rate of the fluid as it collects in the container. The method also includes calculating a volume of the fluid as it collects in the container based on data measured from the capacitance sensor. The measured data from the capacitance sensor is representative of a capacitance of the capacitance sensor, and the volume is calculated based on the measured data representative of the capacitance of the capacitance sensor. The measured data representative of a capacitance of the capacitance sensor may be measured indirectly from the changing frequency of an oscillator, or by using a CVD, a Bridge method, a Charge-Based method, and/or a CSM method.

The method may also involve calculating a base capacitance of the capacitance sensor prior to calculating a volume of the fluid, so that the change in capacitance due specifically to the liquid can be identified and the volume more accurately calculated. The base capacitance may be set to zero, so only the capacitance of the fluid is measured.

In one embodiment, a high resolution, low cost inline flow meter device for measuring urine production of a patient carrying a urine catheter is provided. The flow meter provides immediate fluid flow readings without columnating or creating obstructions within the drain lumen. This is an advantage over current technologies and methods. This embodiment also provides an automatic, low power device for calculating, measuring, storing and displaying the urinary flow rates.

In one embodiment, a flow meter includes a housing including a fluid passage therethrough, and a capacitance sensor inside the housing configured to act as a capacitor to sense a physical property of the fluid as it passes through the fluid passage. The flow meter also includes a microcontroller programmed to calculate a volume of the fluid as it passes through the fluid passage based on a measurement from the capacitance sensor. The flow meter may also include a wireless transceiver for transmitting the measured/calculated data, including volume and flow rate, to a separate device (e.g., a computer, monitor, smart phone, etc.). The capacitance sensor of the flow meter may have a coaxial electrode structure disposed around the fluid passage, or an electrode structure including two semicircular plates, the fluid passage disposed between the two semicircular plates. The flow meter may also include a superhydrophobic microstructure patterned surface formed on an inner surface of the fluid passage.

In one embodiment, the lumens of the tubing/catheters, etc. of the system are coated or treated with a surfactant to reduce unwanted fluid within bladder and drainage lumen and prevent columnation. This embodiment provides immediate fluid flow without columnating within the drainage lumen/bladder to overcome any surface tension forces introduced by the drainage lumen.

In one embodiment, the lumens of the tubing/catheters, etc. of the system are formed with a superhydrophobic patterned surface to reduce unwanted fluid within bladder and drainage lumen and prevent columnation. This embodiment provides immediate fluid flow without columnating within the drainage lumen/bladder to overcome any surface tension forces introduced by the drainage lumen.

In one embodiment, a urine monitoring system, includes a container for collecting urine, a printed electronic resistive sensor attached to an internal surface of the container and configured to measure a physical property of the urine as it collects in the container, and a microcontroller programmed to calculate a volume of the urine as it collects in the container based on a measurement from the printed electronic resistive sensor.

In one embodiment, a urine monitoring system includes a container for collecting urine, a force-sensing resistor configured to provide a measurement value indicative of volume of the urine as it collects in the container, a support and measurement assembly from which the container hangs, the support and measurement assembly including a contact object disposed directly above and in contact with the force-sensing resistor; and a microcontroller programmed to calculate a volume of the urine as it collects in the container based on the measurement value from the force-sensing resistor.

### BRIEF DESCRIPTION OF THE DRAWINGS

The disclosed systems and methods can be better understood with reference to the following drawings. The components in the drawings are not necessarily to scale.

FIG. 1 shows a front view of a capacitance-based fluid measurement or monitoring device/system.

FIG. 2 shows a back view of the capacitance-based fluid measurement or monitoring device/system of FIG. 1.

FIG. 3 shows a capacitance-based fluid measurement or monitoring device/system having two fringing field, parallel strip/plate electrodes on a flexible collection bag.

FIG. 4 shows a capacitance-based fluid measurement or monitoring device/system having two fringing field, parallel strip/plate electrodes on a rigid blow molded collection container.

FIG. 5 shows a capacitance-based fluid measurement or monitoring device/system having a fringing field, interdigital electrode structure on a flexible collection bag.

FIG. 6 shows a capacitance-based fluid measurement or monitoring device/system having a fringing field, pseudo interdigital electrode structure on a flexible collection bag.

FIG. 7 shows a capacitance-based fluid measurement or monitoring device/system having a parallel plate electrode structure with electrodes attached to opposite facing walls of a rigid fluid collection container.

FIG. 8A shows a capacitance-based fluid measurement or monitoring device/system having a parallel plate electrode structure with electrodes attached to opposite facing rigid walls, the other walls being flexible and expandable.

FIG. 8B shows a side view of the capacitance-based fluid measurement or monitoring device/system of FIG. 8A.

FIG. 9 shows a capacitance-based fluid measurement or monitoring device/system having a parallel plate electrode structure with electrodes disposed within a rigid fluid collection container.

FIG. 10A shows a capacitance-based fluid measurement or monitoring device/system in the form of an inline flow meter arranged in line with a Foley catheter.

FIG. 10B shows a cross sectional view of the inline flow meter of FIG. 10A as a semicircular parallel plate capacitance sensor.

FIG. 10C shows a cross sectional view of the inline flow meter of FIG. 10A as a coaxial capacitance sensor.

FIG. 11 shows a coaxial ring-type capacitor.

FIG. 12 shows a relaxation oscillator internal microcontroller.

FIG. 13 shows a relaxation oscillator Schmitt Trigger.

FIG. 14 shows a Capacitive Voltage Divider technique for measuring capacitance.

FIG. 15 shows a Bridge AC excitation approach to measuring capacitance.

FIG. 16 shows a charge transfer method for measuring capacitance.

FIG. 17 shows a microchip microcontroller internal capacitive sensing module.

FIG. 18 shows a capacitive sensing module block diagram.

FIG. 19 shows liquid droplets sitting on top of a rough superhydrophobic patterned surface.

FIG. 20 shows a superhydrophobic microstructure patterned surface formed on a portion of the inner surface of a catheter/tubing (not to scale).

FIG. 21 shows a fluid monitoring device or system implementing a printed electronic resistive sensor.

FIG. 22 shows simplified circuit diagram of a reliable, low cost fluid monitoring device or system implementing a printed electronic resistive sensor.

FIG. 23 shows simplified block diagram of hardware of a fluid monitoring device or system implementing a printed electronic resistive sensor.

FIG. 24 shows a fluid monitoring device or system implementing a Force-Sensing Resistor (FSR).

FIG. 25 shows some of the components of a Force-Sensing Resistor (FSR).

FIG. 26 shows a design of a mechanical fixture for holding the Force-Sensing Resistor (FSR) sensor contact area constant and preventing bending.

FIG. 27 shows simplified circuit diagram of a fluid monitoring device or system implementing a Force-Sensing Resistor (FSR).

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DESCRIPTION

The following description and accompanying figures, which describe and show certain embodiments, are made to demonstrate, in a non-limiting manner, several possible configurations of a reliable, low cost fluid (e.g., urine) monitoring apparatus and/or system, including for measuring volume and flow rate, according to various aspects and features of the present disclosure. The devices and systems disclosed may be used as urine monitoring devices/systems, or may be used to monitor other fluids in various applications. Additionally, the disclosure relates to ways of improving fluid flow through the system thereby improving measurements and helping to prevent unwanted fluid from remaining in the system.

As used herein, the term “accuracy” refers to a measure of rightness, e.g., the agreement between a measurement and the true or correct value. While accuracy refers to the agreement of the measurement and the true value, it does not tell you about the quality of the instrument used. “Error” refers to the disagreement between a measurement and the true or accepted value. “Precision” is a measure of exactness and refers to the repeatability of measurement. “Resolution” refers to the minimal change of the input necessary to produce a detectable change at the output. “Transducer” refers to a device that transfers energy between two systems as in the conversion of thermal into electrical energy by the Seebeck-effect thermocouple. The words “including,” “has,” and “having,” as used herein, including the claims, shall have the same meaning as the word “comprising.”

The reliable, low cost fluid (e.g., urine) monitoring devices and/or systems of this disclosure include without limit capacitance-based measurement systems, pressure-based measurement systems, weight-based transducer measurement systems (e.g., load cell or strain gage systems), and/or other low cost, high resolution measurement systems.

### Capacitance-Based Measurement Systems

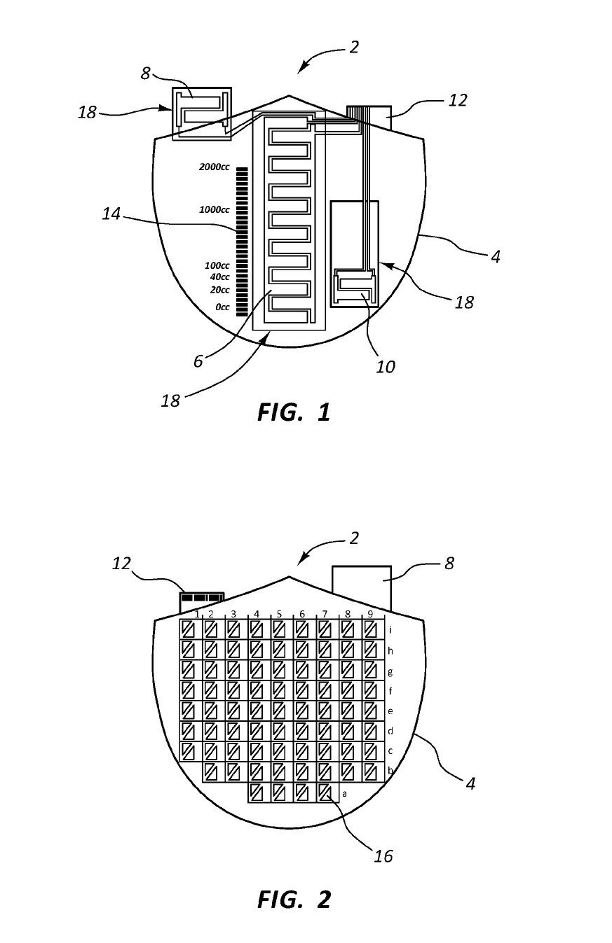

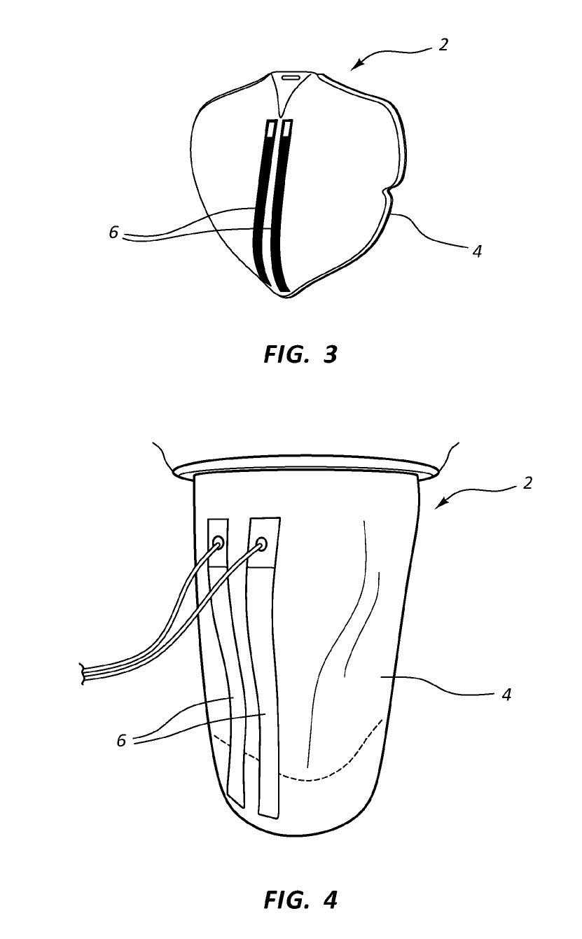

FIGS. 1 and 2 show front and back views, respectively, of a high resolution, low cost fluid monitoring device or system in the form of a capacitance-based fluid measurement device/system. The device/system shown in FIGS. 1 and 2 is exemplary of a capacitance-based fluid measurement device/system. While the device/system of FIGS. 1 and 2 is generally referred to herein as urine monitoring system or urine meter 2, the general principles and features disclosed may be applied to a wide variety of forms and applications of capacitance-based fluid measurement/monitoring devices and/or systems, and may be used to monitor fluids other than urine.

Smart urine meter 2 uses a capacitance sensor 6, which operates using capacitance-based measurement principles. Capacitance sensor 6 behaves like an electrical capacitor that is acted on by the amount of fluid/urine in the container, and whose capacitance is influenced by the time-dependent amount of fluid/urine present. The fluid/urine acts both as an electrical conductor and as a dielectric, and the capacitance is used as an indication of the filled volume function, which is differentiated electrically. Changes in volume are also tracked over time to monitor flow rate. Accordingly, capacitance sensor 6 can measure volume and flow rate.

Generally, a capacitor consists of at least two electrodes (e.g., conducting plates). The electrodes may be separated or influenced by a substance called a dielectric. Capacitance is the measure of the amount of charge that a capacitor can hold at a given voltage. Capacitance is measured in Farads (F) and it can be defined in the unit coulomb per volt as:

$$C = \frac{Q}{V} .$$

Permittivity is a physical property of matter, and is important in the design and construction of capacitors. The permittivity of a vacuum (also known as free space) is equal to approximately 8.85 pF/m. The dielectric constant K or relative permittivity of a material/substance is the ratio of the permittivity of the material/substance to the permittivity of free space. In other words, the dielectric constant K of a material is the ratio of the permittivity of the medium (∈ᵣ) to the permittivity of free space (∈₀) (i.e., a vacuum, or air as a very close approximation); ∈₀=8.85 pF/m. Free space has a dielectric constant of 1, and most substances have a dielectric constant greater than this. Water has a high dielectric constant of 80.10 at 20° C.

In general, the capacitance of a capacitor is determined by the area of each electrode, the distance between the electrodes, and the permittivity of the dielectric material. The capacitance of a capacitor can be expressed in terms of its geometry and dielectric properties as

$$C = \frac{( {ɛ}_{0} {ɛ}_{r} A )}{d}$$

(where C=capacitance in farads (F), ∈₀=the permittivity of free space (8.854×10⁻¹² F/m), ∈ᵣ=the relative permittivity or dielectric constant, A=effective area (square meters), and d=effective spacing (meters)). The capacitance phenomenon is related to the electric field between the electrodes of the capacitor. Voltage is applied to the electrodes, and the impedance across the electrodes, which changes due to capacitance variations, can be measured and correlated to changes in volume and/or flow rate.

As shown in FIGS. 1 and 2, smart urine meter 2 may comprise a fluid collection container/bag 4, capacitance sensor 6, a reference capacitor 8, a compensation capacitor 10, a finger-type card edge connector 12, a reference scale 14, an electric field sensor matrix 16, and rigid or semi-rigid panels/surfaces 18.

A wide variety of types of fluid collection containers or bags may be used for fluid collection container 4. For example, container 4 may be similar to any known urine collection container or urine collection bag. Container/bag 4 may take a variety of sizes, shapes, and forms and may be flexible, rigid, semi-rigid, or a combination of these. Indeed, capacitance sensor 6 can measure volume and flow rate arbitrary of the size or shape of the bag/container. However, rigid or semi-rigid materials beneficially help minimize variation on the capacitive electrodes of capacitance sensor 6.

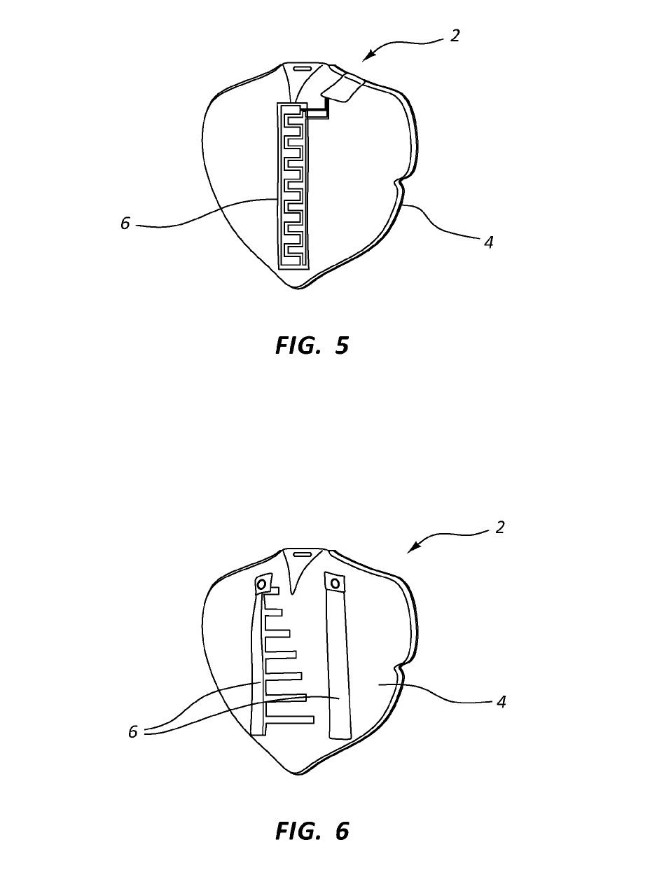

Container 4 may be formed with a variety of types of materials known to be suitable for urine collection bags/containers. For example, container 4 may be formed with a thin PVC structure (as depicted in FIGS. 3, 5, and 6), may be a more rigid blow molded plastic container (as depicted in FIG. 4), or may be a container that combines rigid materials and flexible materials (as depicted in FIGS. 8A and 8B). Container 4 can be shaped and sized for various different applications. In some instances, container 4 will be between about 7-15 inches in height and about 1300-3000 mL in volume, (e.g., the container 4 may be about 10 inches in height and about 2000 mL in volume). Preferably, container 4 has a large enough volume to collect at least the average volume of urine produced by the average patient during urination. Typically, a container volume of at least 2000 mL is desired. Different sizes may be used for different applications, e.g., urine collection from small children may involve smaller sizes than urine collection from adults.

In practice, the container 4 may be designed to fill with fluid from the top or the bottom of the container, e.g., urine can flow from a Foley catheter into tubing associated with the container that empties into the container 4. In one embodiment, fluid flows through tubing connected at the top of container 4 to fill the container 4. The fluid generally flows through the tubing/catheter into the container 4 due to gravitational force (although urine may in some circumstances be drained by other forces, e.g., via a pump). Container 4 may also include a component for removing measured quantities of urine for various testing procedures or merely for emptying the container 4, such as a drainage tube, drain port, and/or drain valve.

Capacitance sensor 6 is a variable permittivity capacitance sensor that forms a sensitive probe in order to measure the volume, flow rate, composition, etc. of a fluid (e.g., urine). Capacitance sensor 6 implements an autonomous inexpensive circuit, and indicates volume and flow rate arbitrary of the size or shape of the bag/container. Capacitance sensor 6 can be regarded as a capacitor. The capacitance of capacitance sensor 6 has a reciprocal relationship with fluid (e.g., urine) content, and can be correlated to and used to measure and calculate the fluid volume, computed flow rate, composition of the fluid, and other parameters. Capacitance sensor 6 is able to measure absolute levels of both conducting and non-conducting liquids. The capacitance sensor 6 is robust and also eliminates the need for factory calibration.

Because the capacitance also depends on the permittivity of the measured fluid, and because capacitance can change with fluid composition, in some embodiments composition measurements can be made. For example, the effect of different materials found in urine may be correlated with their effect on the capacitance of the sensor to give an indication of composition of the urine. In its broadest form, the capacitance measurements may merely give an indication that elevated levels of a particular component of the urine exist, and may trigger a warning if the levels are dangerously high. With increased sensitivity, capacitance sensor 6 will be able to give more precise indications of composition.

Capacitance sensor 6 can be integrated with container 4 (e.g., attached to an inner or outer side wall) or can be inserted into container 4 without direct attachment to the walls of container 4. Capacitance sensor 6 does not need to be in physical contact with the urine, which allows capacitance sensor 6 to detect/measure urine or other fluids through nonconductive materials, e.g., through the plastic sides of the container. In FIG. 1, capacitance sensor 6 is shown as being attached to a clear semi-rigid panel/surface 18 on the outside of the container 4 (panel/surface 18 may be attached to or integral with container 4) to help minimize variations on the electrodes, but other means of attachment or integration with container 4 are also possible.

Capacitance sensor 6 may also be formed from a conductive ink layer printed on, painted on, or otherwise applied to the side of container 4 (or to a another surface that is attached to the side of container 4, e.g., a semi-rigid panel/surface), with the conductive ink forming the electrodes of the capacitor and being spaced a fixed distance apart. For example, the conducting layers of the capacitor can made of thin nickel conductive based ink, graphite based conductive ink, or silver based conductive ink. Optionally, the electrodes can be formed using strips or plates of conductive material, or conductive tape (e.g., copper tape). Also, a metalized type of paper may be patterned to create electrode pads of arrayed mesh. Each of the above electrode types is relatively inexpensive and provides for a low-cost capacitance sensor 6, and an overall low-cost urine meter 2.

In FIG. 1, capacitance sensor 6 is shown as having a low-cost coplanar electrode structure formed from interdigitated electrodes (see also FIG. 5). An interdigital capacitor/sensor electrode structure is formed when multiple electrodes are stacked in parallel a fixed separation distance apart, and every other stacked electrode is electrically connected together.

Fringing field interdigitated (e.g., as shown in FIGS. 1 & 5), and parallel strip/plate (e.g., as shown in FIGS. 3 & 4), and pseudo interdigitated (e.g., as shown in FIG. 6) electrode structures use the same principle of operation as two-sided parallel plate or cylindrical coaxial capacitors. However, unlike the parallel-plate cell with two facing plates, the fringing field capacitance sensors do not require two-sided access to the material under test. Indeed, these fringing field electrode structures may be coplanar or generally coplanar (e.g., mostly coplanar with minor variations due, for example, to the contours of the container). Indeed, the electrode structures are not necessarily entirely coplanar, e.g., the electrode structures may bend with the contours of container 4. In fringing field capacitors, fringing electric field lines arc (similar to a semicircle or arch) up from one electrode up through the material under test and back to another parallel electrode. Because the electric field lines arc through the material, the capacitance and conductance between the two electrodes depends on the material's dielectric properties as well as on the electrode and material geometry. The capacitance becomes a function of the liquid properties. Therefore by measuring the capacitance of the sensor, the system and liquid properties can be evaluated. Other capacitance measurements could also be used i.e. resistor or capacitance voltage discharge.

The design and geometry of the electrode structure of capacitance sensor 6 may vary depending on the desired properties of the sensor and on the intended application (see e.g., FIGS. 1-10 showing, without limitation, various electrode configurations that may be used). Capacitance sensor 6 may be formed with a fringing field electrode structure, or may be formed using non-planar electrodes (e.g., parallel plate) that do not rely on (i.e., are not dependent upon) fringing fields. The geometry of the sensing electrodes influences the electric field between them. A time-dependent electrical and mechanical model can be easily used to tailor the characteristics of capacitance sensor 6 to the particular application and/or arrangement for which it is used. In one embodiment, the capacitance sensor 6 will have a measuring range of about 2000 mL with 5% accuracy. The capacitance sensor 6 preferably functions over a large temperature range, e.g., from −25° C. to +75° C.

In one embodiment, capacitance sensor 6 is an interdigital capacitor/sensor with the following dimensions: thickness of the electrodes=about 200 mm; distance between adjacent parallel electrodes=about 1 mm; distance between the center of adjacent parallel electrodes=about 2 mm; width of each parallel electrode=about 2 mm; length of each parallel electrode=about 20 mm; and number of parallel electrodes=22.

In one embodiment, capacitance sensor 6 is a fringing field parallel strip capacitor/sensor with two parallel electrodes fabricated to be about 200 mm long and about 9 mm wide, and have a separation distance of about 5 mm. The electrodes may be coplanar or generally coplanar (e.g., mostly coplanar with minor variations due, for example, to the contours of the container).

Once the size/configuration of capacitance sensor 6 and the dielectric material are fixed, the permittivity of the measured medium (e.g., urine) can be analyzed from the capacitance. For n interdigitated electrodes, the capacitance can be approximated as follows:

$$C = \frac{( n - 1 ) {∈}_{0} {∈}_{r} A}{d} .$$

As can be seen, if a capacitor is constructed with n number of parallel plates, the capacitance will be increased by a factor of (n−1). If only two parallel strips are used as electrodes, instead of multiple interdigitated electrodes, then (n−1) equals 1. For simplicity, this equation does not account for multiple materials with different relative permittivity values. However, as discussed below, reference capacitor 8 and compensation capacitor 10 can be used to account for variations in the composition of the fluid.

To prevent short-circuiting of the input of the measurement system (e.g., when used with conducting liquids like water or urine), the electrodes may be covered with an insulating material, e.g., as a coating, additional layer, or sleeve (not shown). This insulating material can also protect the electrodes against an aggressive environment, e.g., in urine. Assuming a coplanar electrode structure, covered with an infinitely thin insulating material, the conducting liquid can be regarded as a shield that is connected to ground. The capacitance between a single electrode segment and the opposite or adjacent electrode can be calculated as a function of the interface level.

Capacitance sensor 6 may include multiple different substrate layers. For example, capacitance sensor 6 may include three layers, including a conducting electrode layer, a shielding layer, and a ground layer. Using multiple layers in this way improves the sensor sensitivity and increases reliability.

Optionally, a reference capacitor 8 and a compensation capacitor 10 may also be included in urine meter 2, e.g., as shown in FIG. 1. Reference capacitor 8 and compensation capacitor 10 are shown in FIG. 1 as being attached to clear semi-rigid panels 18 on the outside of the container 4 (panel/surface 18 may be attached to or integral with container 4) to help minimize variations on the electrodes, but other means of attachment or integration with container 4 are also possible. By having reference capacitor 8 separated from the fluid being measured and instead exposed to the air, reference capacitor 8 acts as a comparative reference that approximates the relative permittivity or dielectric constant of free space (i.e., 1). In contrast, compensation capacitor 10 is exposed to the fluid such that its capacitance is affected by the relative permittivity of the fluid. A microcontroller can be programmed to process data from the reference capacitor and compensation capacitor to detect and compensate for any dielectric changes in the fluid in real time, e.g., if the composition of the measured liquid varies over time. Changing composition can change the overall dielectric constant of the liquid and alter the resultant capacitance generated potentially causing error in the measurements. Also, large variations in the conductivity of the measured material over time can potentially cause error in measurements. Reference capacitor 8 and compensation capacitor 10 can help compensate for these variations by determining the dielectric constant of the measured liquid in real time, thereby eliminating or reducing the error that might otherwise be caused by such variation in the measured liquid.

Any data measured by the urine meter 2 (e.g., volume and flow rate data) may be sent to another device or computer (e.g., C.R. Bard's Criticore® monitor or similar monitors, a desktop, a laptop, a smart phone, etc.) to collect, process, and/or store the data for review and tracking. A finger-type card edge connector 12 as shown in FIG. 1 may optionally be included as part of urine meter 2. The finger-type card edge connector 12 provides a means of connecting urine meter 2 to another device or computer, and provides a means for communicating data from capacitance sensor 6, reference capacitor 8, and compensation capacitor 10 to the connected device or computer.

Other devices, systems, or means for connection/communication between urine meter 2 and other devices or computers are also possible. For example, urine meter 2 may include a USB port, and/or may be tethered to a device or computer through a wired connection. Alternatively, urine meter 2 may include a wireless transmitter or transceiver (e.g., Zigbee, etc.) to transmit data wirelessly. In one embodiment, short range radiofrequency (RF) principles may be used. Some short range RF protocols that can be used are referred to as “Bluetooth.” Wireless 802.11 communication principles and/or similar communication principles may also be used. Urine meter 2 or the device or computer with which it communicates may optionally be connected to a network (e.g., the internet or a local network) and the data may be shared with and/or processed by other devices or computers connected to the network.

In one embodiment, multiple urine meters each connected to a different patient are configured to transmit data to the same computer or network. This allows tracking and/or comparing data from multiple patients at a single location. Software associated with each urine meter can be programmed to transmit the measured data with a unique identifier to distinguish the data transmitted by one urine meter from the data transmitted by each of the other urine meters.

As shown in FIG. 1, a reference measurement scale 14 may optionally be provided on the inner or outer surface of container 4. Reference scale 14 includes graduated markings based on volume and allows for visual confirmation or reading of the volume of liquid in container 4.

As shown in FIG. 2, urine meter 2 may optionally include an electric field sensor matrix 16. Electric field sensor matrix 16 is shown in FIG. 2 as being attached to the outside of the container 4. Electric field sensor matrix 16 can be attached to a clear semi-rigid panel/surface (attached to or integral with) container 4 (e.g., similar to semi-rigid panels/surfaces 18), this helps minimize variations on the electrodes. but other means of attachment or integration with container 4 are also possible (e.g., printing the electrodes directly on the surface of container 4). Electric field sensor matrix 16 can be used to detect tilt in urine meter 2. This helps prevent errors in volume measurement that may arise from the measured liquid not being properly aligned with capacitance sensor 6 due to container 4 being tilted. The electric field sensor matrix 16 may trigger an alarm or other warning telling the practitioner to realign container 4 to correct the tilt. Alternatively, data from electric field sensor matrix 16 may be used in calculations to compensate for any tilting effects. Electric field sensor matrix 16 may be formed from a series of relatively small electrodes on a side of urine meter 2 (or on another surface attached to a side of urine meter 2, e.g. a semi-rigid panel similar to panel/surface 18 shown in FIG. 1). The matrix of relatively small electrodes may be formed with materials similar to those used for capacitance sensor 6, e.g., the electrodes may be printed or painted on urine meter 2 using conductive ink. Also, the electrodes may operate on a capacitance-based principle similar to capacitance sensor 6.

Urine meter 2 may include a microcontroller and integrated circuit connected to capacitance sensor 6, reference capacitor 8, compensation capacitor 10, a wireless transceiver, electric field sensor matrix 16, etc. Finger-type card edge connector 12 may be formed on the edge of the integrated circuit or otherwise connected. The microcontroller and/or integrated circuit may include a relaxation oscillator, analog to digital converter, or other features for measuring capacitance (e.g., any features discussed below in the discussion(s) regarding measuring capacitance). Also, any circuits belonging to the class of intelligent capacitance measuring circuits may be used in urine meter 2. For example, a discrete oscillator circuit (e.g., a cd4060 circuit) may be used. Alternatively, an integrated circuit like the Universal Transducer Interface (UTI) can be used. In one embodiment, a stable oscillator for the sensor circuit and a microcontroller for the signal processing are used.

In the microcontroller, the measurement data from the sensor 6 and data from the other features discussed above are processed by written software or firmware. This software/firmware consists of functions which combine the measurements data to produce usable quantities for the user. For example, the measured capacitance of capacitance sensor 6 may be correlated to volume level using linearization and/or curve-fitting procedures. The software/firmware may then be programmed with the relationship in order to calculate for any given capacitance measurement, a value for liquid volume in container 4. The software/firmware may also be programmed to track the volume level over time to calculate flow rate. The software/firmware may also signal that the volume level, flow rate, and any other measured/calculated parameters be displayed on or transferred to a monitor, computer, smart phone, and/or other device. The parameters may be continuously calculated, updated, and displayed in real time, e.g., during urine collection. The software/firmware may also be programmed to accomplish other purposes/functions, including those discussed elsewhere herein.

In one embodiment, the microcontroller is a 32-bit PIC 32 microcontroller. The PIC32 board provides a complete, high-quality development platform for PIC32MX7 series devices. It has numerous on-board modules (Ethernet PHY), I2C, SPI, RTC, audio codec, accelerometer, temperature sensor, and flash memory, which allows to write applications of high complexity quicker.

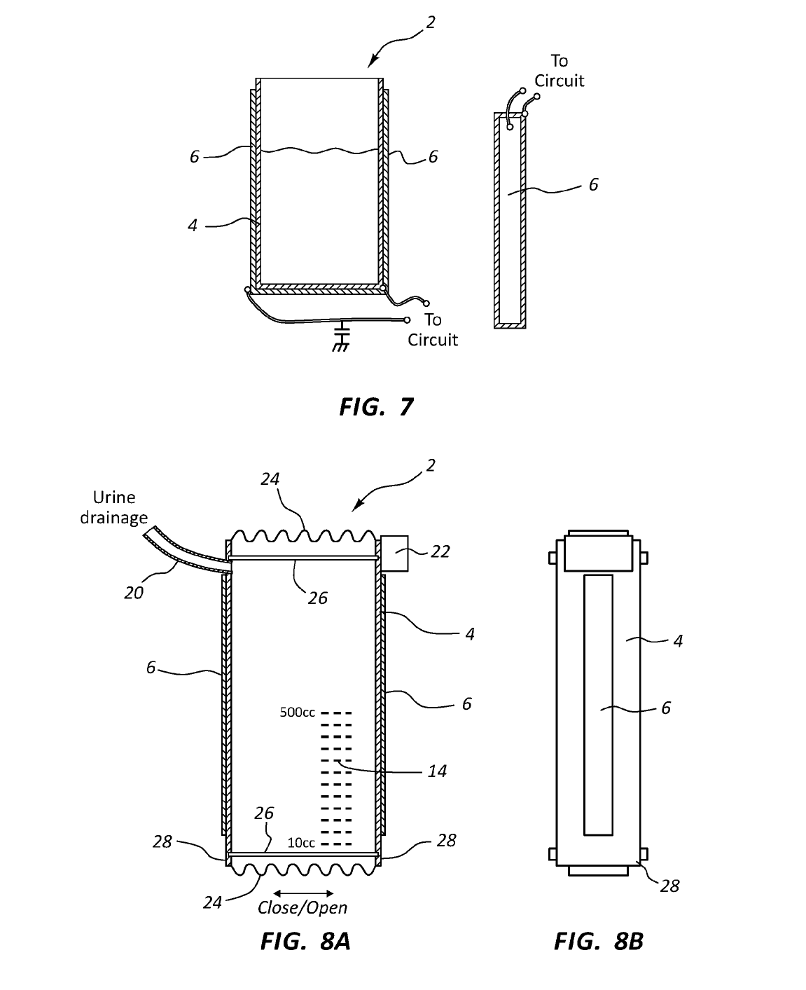

Structural variations in urine meter 2, including structural variations in capacitance sensor 6, are possible without straying from the general principles described herein, e.g., capacitance-based sensing principles. For example, in one embodiment, as shown in FIG. 7, parallel plate electrodes form or are attached to opposite facing walls of the fluid collection container 4, such that capacitance sensor 6 provides two sided access to the material under test. The walls with the electrodes are rigid and set a fixed distance apart to eliminate or minimize variances on the electrode distance. The other walls not including the electrodes may be rigid or flexible. In one variation, the walls of container 4 that do not include an electrode are flexible and accordion shaped. These flexible walls spread or flex to accommodate the fluid, while the walls including the electrodes remain a fixed distance apart. This embodiment functions similar to the other capacitance-based meters described herein, and may include the additional features disclosed in the various embodiments discussed herein whether or not shown on FIG. 7.

In one embodiment, as shown in FIGS. 8A and 8B, the walls 28 of container 4 that include the capacitance sensor 6 electrodes are rigid, whereas the portions/walls that do not include an electrode are flexible and/or accordion shaped, e.g., flexible portions 24 (which can be made of a thin plastic material). The flexible walls spread or flex to accommodate the fluid. In this embodiment, the distance between the electrodes is allowed to fluctuate within an acceptable range as the urine meter fills from urine drainage tubing 20. The distance between the electrodes can be measured automatically, e.g., by tracking the expansion of extension bars 26 or by using other means to measure and compensate for variations in distance. The software/firmware may be programmed to track and compensate for changes in the distance between electrodes. Otherwise, this embodiment functions similar to the other capacitance-based meters described herein, and may include the additional features disclosed in the various embodiments discussed herein whether or not shown on FIGS. 8A and 8B. A wireless transceiver 22 is shown, which functions similar to the other wireless transceivers discussed herein.

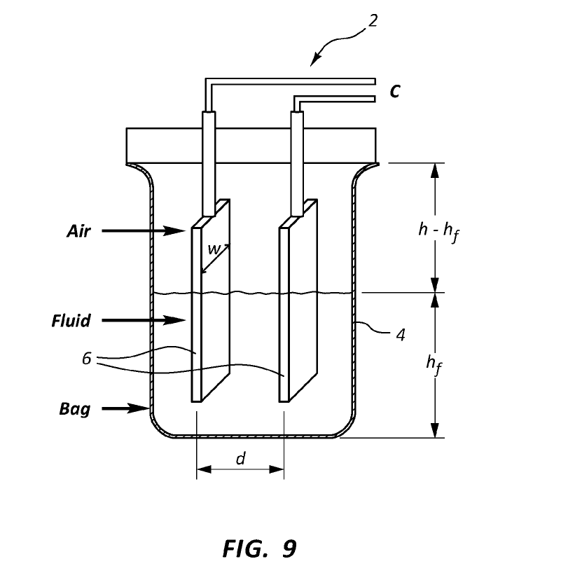

In one embodiment, as shown in FIG. 9, parallel plate electrodes are inserted within the fluid collection container 4 such that they face each other to form the capacitance sensor 6. The parallel plate electrodes may be attached/connected to the top or lid of the container 4. The parallel plate electrodes are attached/connected such that they remain a fixed distance apart within a tolerance. This embodiment functions similar to the other capacitance-based meters described herein, and may include the additional features disclosed in the various embodiments discussed herein whether or not shown on FIG. 9.

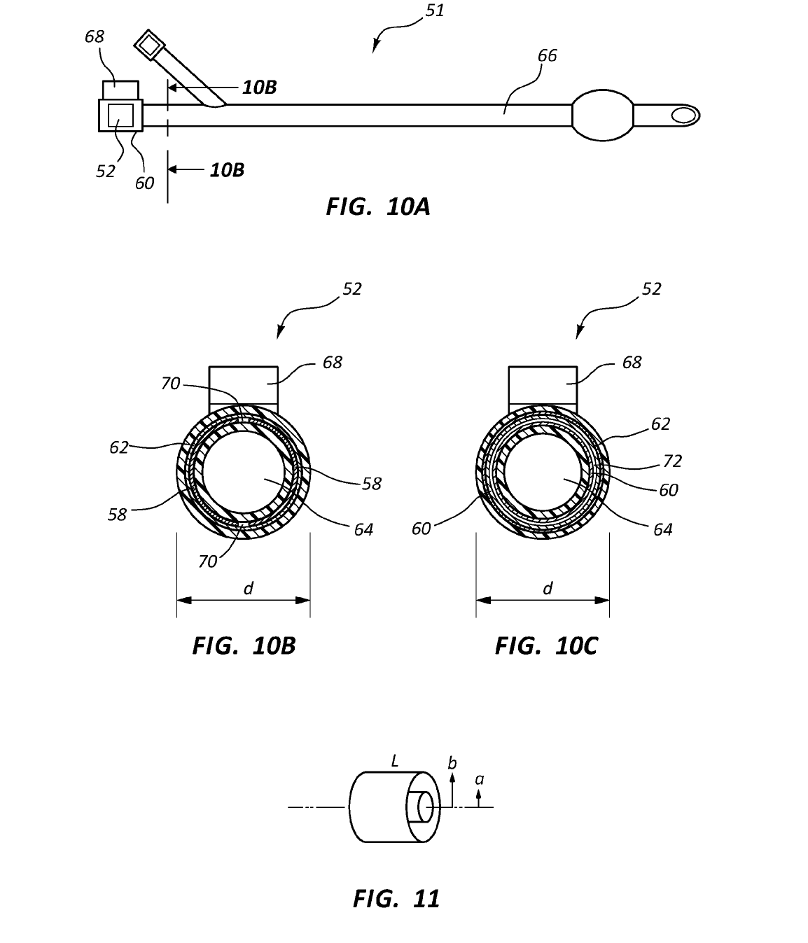

FIGS. 10A-10C show one embodiment of a flow meter 52 that operates based on a similar capacitance measurement-based principle as urine meter 2, but does not collect fluid. Instead flow meter 52 measures the flow rate, volume, composition, etc. as the fluid (e.g., urine) passes through the housing of the device. Flow meter 52 can be arranged in line with a Foley catheter 66 (or other tubing conveying a fluid) to form a fluid measurement assembly 51, e.g., as shown in FIG. 10A. Flow meter 52 measures fluid as it flows through the central lumen of flow meter 52, entering flow meter 52 from the catheter/tubing and exiting flow meter 52 into additional tubing and/or a fluid collection/disposal container or unit. Because flow meter 52 does not itself collect fluid/urine, this flow meter can be much smaller in size than urine meter 2 discussed above (see e.g. FIG. 1).

Flow meter 52 provides a means for measuring the urine production of a patient and the dielectric change. Flow meter 52 can provide an immediate value of the current flow rate, providing a faster and more direct response than current technologies. Flow meter 52 could also be configured to measure the chemical composition or the concentration of electrolytes in a fluid or urine, e.g., based on the dielectric change.

The embodiment shown in FIGS. 10A-10C includes a capacitance sensor for measuring duration, volume, flow rate, composition, etc. As shown in the two cross sections of FIGS. 10B and 10C, the capacitance sensor 56 may be formed as a semicircular parallel plate capacitor formed from two semi circular metalized parallel plates 58 disposed on opposite sides of the fluid passage (the plates 58 include spaces 70 or an insulator between them), or a coaxial ring-type capacitor formed from two concentric, coaxial cylindrical ring electrodes 60 (see also FIG. 11 showing a coaxial ring-type capacitor with concentric ring electrodes). Coaxial ring electrodes 60 can be spaced apart by a space or insulator 72 (FIG. 10C is not necessarily to scale and the spacing/thickness may be different, e.g., larger, than shown). The formula for capacitance of a semicircular parallel plate capacitor is C=(E A)/d), while the formula for capacitance of a coaxial ring capacitor is C=[(2π∈ₒ∈ᵣ)/ln (b/a)]*L. As shown in FIG. 11, “b” is the radius of the outer coaxial electrode, “a” is the radius of the inner coaxial electrode, and “L” is the length of the electrodes. Capacitance sensor 56 may be surrounded by an electromagnetic interference (EMI) shield 62 to reduce external interference or noise. Other methods for improving the quality of capacitance measurements, as discussed below, may also be used.

Outside of the EMI shield 62 is the outer region of the flow meter housing, while the inner region of the housing forms the surface of fluid passage 64. Optionally, the wireless transceiver, microcontroller, and other circuitry may be included within the housing of the flow meter, or may be attached to the outside portion of the housing of the flow meter.

The fluid being measured (e.g., urine) flows through the central inner lumen or fluid passage 64 of capacitance sensor 56. The central inner lumen or fluid passage 64 of capacitance sensor 56 has a diameter that is approximately the same diameter as the tubing or Foley catheter 66 to which it is connected so as not to interrupt or change the flow rate of the fluid flowing through the tubing/catheter. Preferably, the inside of capacitance sensor 56 and the associated tubing/catheter is coated with a hydrophobic coating and/or includes a superhydrophobic pattern design to reduce urine surface tension on the sensor and the tubing/catheter, as discussed in more detail below. This provides a better emptying mechanism and prevents fluid from being held for too long within the sensing area thus affecting the readings.

Capacitance sensor 56 can easily measure the duration of urination because the capacitance of capacitance sensor 56 will suddenly change when the first urine enters the fluid passage 64 of flow meter 52, and the capacitance will also change by a significant amount when the last of the urine leaves the fluid passage 64 of flow meter 52. The volume of the urine can also be estimated because the volume of fluid passage 64 is known in advance. The amount of capacitance registered by capacitance sensor 56 will correspond to how full the fluid passage 64 is as the urine passes through. This can be used to estimate the volume of fluid passing through fluid passage 64 at any given time. Alternatively, urine volume can be measured in the final collection container (e.g., urine meter 2 or a volumetric collection container) and be processed in combination with the duration of urination measured by capacitance sensor 56 to calculate flow rate. Additionally, according to one embodiment, the capacitance sensors may be arranged in a series along a length of fluid passage 64 and, based on a Doppler theory, measure the amount of time it takes for a bolus of fluid to move along that length and/or from sensor to sensor.

The capacitance of capacitance sensor 56 may be measured using any method of measuring capacitance discussed below. Also, flow meter 52 may include a microcontroller and/or integrated circuit similar to those used with urine meter 2 that can include firmware/software, e.g., to monitor and analyze the timing and the intervals of urine excretion to detect real time hourly flow rate values, and to track accumulated values.

Flow meter 52 may also include a wireless transceiver 68, similar to the wireless transceivers discussed with respect to urine meter 2 (e.g., Zigbee, etc.), to wirelessly communicate with a remote computer or unit to improve the usability of the system. Alternatively, flow meter 52 may include one of the other means of communication disclosed above with respect to urine meter 2. With respect to data transmission, flow meter 52 can function in the same way as urine meter 2.

Further, multiple flow meters each connected to a different patient can also be configured to transmit data to the same computer or network as discussed above with respect to urine meter 2. Software/firmware associated with each flow meter can be programmed to transmit the measured data with a unique identifier to distinguish the data transmitted by one flow meter from the data transmitted by each of the other flow meters.

### Capacitance Measurement Methods

To measure the capacitance of a capacitance sensor (e.g., one of the capacitance sensors discussed above), one may use several different methods. The examples described below generally involve using a microcontroller to indirectly measure capacitance. Each method has certain benefits and can be used depending on microcontroller capabilities. Some methods that may be used include: (1) using a Capacitance-Controlled Oscillator, (2) using a Capacitive Voltage Divider (CVD), (3) a Bridge method, (4) a Charge-Based method, and/or (5) a Capacitive Sensing Module method. As used in this disclosure, the terms “measure,” “measures,” “measured,” and “measuring” (e.g., measuring capacitance, measuring permittivity, measuring volume, measuring flow rate, etc.) includes indirectly measuring a parameter (e.g., identifying/calculating the value of capacitance, permittivity, volume, flow rate, etc. based on a measured change in voltage, frequency, etc.). These methods, which are described in more detail below, can be used with any of the capacitance-based meters described herein.

As an initial matter, before measuring fluid output and before any fluid enters the fluid meter, one must account for the base capacitance. The term “base capacitance” refers to the measurement result of an uninfluenced sensor element or an “empty” container (i.e., the capacitance before any of the fluid to be measured is introduced to the capacitance sensor). The base capacitance may be set to a zero value for measurement purposes, i.e., so only the increase or change in capacitance due to fluid collection is measured. The base capacitance should be accounted for or set to zero immediately prior to fluid collection and measurement. This can be done using a button or switch associated with urine meter 2 or another device in communication with the urine meter 2 (e.g., a monitor similar to a Criticore® monitor), whether wirelessly connected or otherwise tethered. The base capacitance button or switch may function similar to a “tare” button that sets a weight scale to zero before a weight measurement. The button or switch can be actuated by the end user (e.g., a clinician) just prior to fluid collection and measurement. Alternatively, urine meter 2 may be configured to set the base capacitance value automatically upon being coupled to another device or monitor (e.g., a monitor similar to a Criticore® monitor) that may be connected to urine meter 2, e.g., to display the measured volume, flow rate, and/or other parameters.

Capacitance-Controlled Oscillator

In one variation, capacitance may be measured using a capacitance-controlled oscillator. For example, capacitance sensor 6 can be connected to a microcontroller/pc and a capacitance-controlled oscillator, e.g., a relaxation oscillator. The oscillator is connected to capacitance sensor 6 such that its frequency is related to or influenced by the capacitance of capacitance sensor 6. The change of the liquid level in container 4 changes the dielectric constant of the combined content of the container 4 (e.g., a combination of liquid and air) causing a frequency change in the oscillator. When there is no fluid, there is little to no capacitance (any residual or base capacitance may be set to zero or otherwise accounted for as discussed above). As soon as liquid reaches the bottom part of the capacitance sensor 6, the capacitance will change the oscillator abruptly to a lower frequency, which begins the measurement range. As the level rises, more capacitance lowers the frequency linearly. At the highest fill level, the lowest frequency will be measured. The change in frequency caused by the change in capacitance is measured by a microcontroller or computer and processed to track volume level over time.

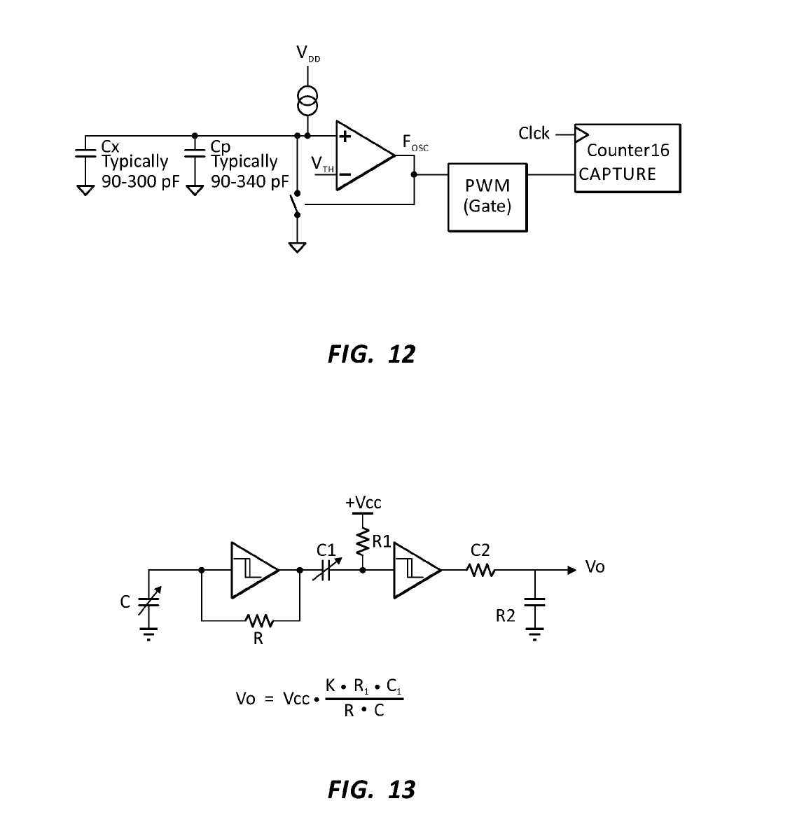

In one method of using an oscillator-based technique to measure capacitance, as depicted in FIG. 12, the internal comparator of a microcontroller is turned into a relaxation oscillator that can be used for capacitive sensing by using the output of the internal comparator to charge and discharge the capacitance sensor 6. The output of the internal comparator will change to the low state. Then, it discharges slowly through R until it reaches the trip point of the internal band gap reference. Following, the output of the comparator will go high again, and the cycle repeats itself. The charge rate is determined by the RC time constant created by an external resistor and the capacitance of the capacitance sensor 6. The output of the comparator is a frequency that is related to the capacitance of the capacitance sensor 6. As the liquid level changes, the frequency changes. As discussed above, this change in frequency is measured by the microcontroller or computer and processed to track volume level.

Optionally, an oscillation circuit may be used. Capacitance is a primary component in determining the frequency of many oscillation circuits. In one embodiment, a 555 timer IC is used as an astable multivibrator. The frequency of oscillation for the 555 timer circuit is given by:

$$= \frac{1.44}{( R 1 + 2 R 2 ) C} .$$

Assuming R1=R2=10K, then C=48000/f, where f is in Hz and C is in nF. In this way, the capacitance is estimated indirectly by measuring the frequency of the 555 output. For example, a 10 ms window can be created in the software, and the number of output pulses within that window can be counted using the timer module (operated as a counter). Assuming N pulses arrive in the 10 ms window, then C=480/N, nF. For example, if N=48, then the measured capacitance would be 10 nF.

In one embodiment, the capacitance sensor 6 is one of the frequency-determining components of a resonant loop, which in turn is part of an oscillator circuit. Capacitance sensor 6 is connected in parallel to an RC-relaxation oscillatory circuit consisting of two inverters i.e. 74HC04, a resistor, Rc, and a capacitor. If the liquid to be measured is brought in the vicinity of capacitance sensor 6, the resonant frequency of the loop changes. The more the capacitance of capacitance sensor 6 is increased by the material under test, the lower the resulting frequency. The microcontroller can be programmed to measure the frequency and then calculate the value of the capacitance from the measured frequency.

Optionally, a CMOS inverter can be used to measure capacitance using a similar oscillator-based technique. The circuit uses a CMOS Schmitt trigger inverter as an RC oscillator followed by a oneshot R1C1 (with a smaller time constant) followed by lowpass R2C2 (with a larger time constant) as seen in FIG. 13. The output can be either capacitance-linear or 1/capacitance linear, depending on the location of the sense capacitor. A floating sense capacitor may be added to increase stability. Again, changes in frequency (as influenced by changes in capacitance) are measured by the microcontroller or computer and processed to track volume level.

In one embodiment, an RC relaxation oscillator is implemented using the IC 555 or its CMOS update, the 7555. This is used to convert capacitance change into a change of frequency or pulse width. The RC oscillator used with a spacing-variation capacitor produces a frequency output which is linear with spacing, while an area-variation capacitor is linearized by measuring pulse width.

The microcontroller clock is usually an accurate and stable reference, and most microcontrollers are therefore able to measure periods or duty-cycles of digital signals over a very large range, a convenient output format of the sensors is period or duty-cycle modulation of square waves. Period modulation has the advantage that only one edge of the signal needs to be monitored, so one can take advantage of interrupt inputs of the microcontroller (when available) that are often either positive or negative edge triggered.

An IC 4060 is an excellent integrated circuit for timing applications. The IC 4060 is an Oscillator, cumulative Binary counter and Frequency divider. Its inbuilt oscillator is based on three Inverters similar to the Schmitt trigger relaxation oscillator. The basic frequency of the internal oscillator is determined by the value of the timing capacitor (Cx) connected to its pin 9 and that of the timing resistor in its pin 10. The IC 4060 has ten active high outputs that can give time delay from few seconds to hours. With a few components, it is easy to construct a simple but reliable time delay circuit. It can be used as a free running timer/frequency divider. Just three external components are required to control the 4060 binary counter, two resistors and one capacitor. The frequency of the internal oscillator (i.e. the speed of the count) is set according to the following equation:

$$f = \frac{1}{2.2 R 1 Cx}$$

Capacitive Voltage Divider (CVD)

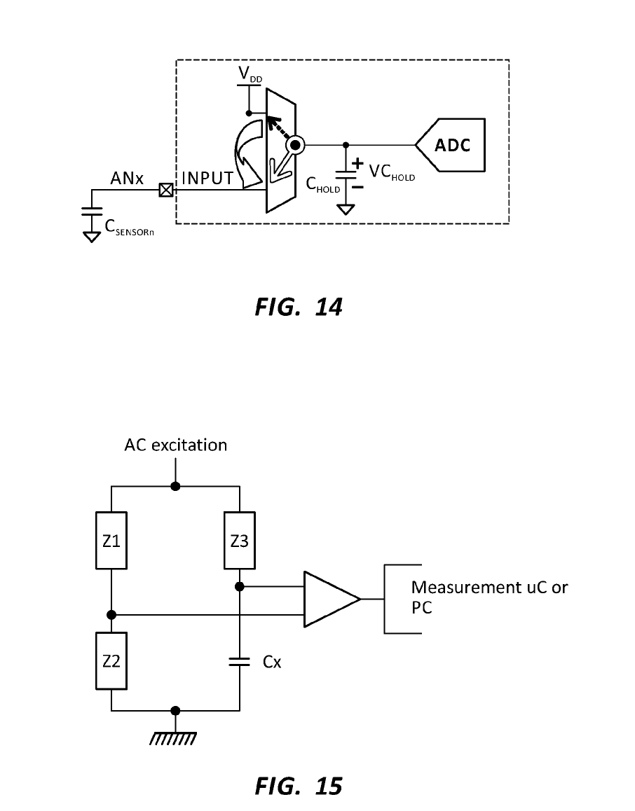

Optionally, methods of measuring capacitance using a CVD may be used. A CVD uses an Analog-to-Digital Converter (ADC) to perform capacitive sensing. The internal sample-and-hold capacitance of the ADC may be used as a reference for calculating sensor capacitance as seen in FIG. 14. The capacitance sensor 6 and the reference capacitor are connected in the circuit, and known values for the reference capacitor and the ADC measurements can be used to identify the capacitance of capacitance sensor 6. Generally, the equivalent capacitance (C_{eq}) of two capacitors connected in parallel is the sum of their capacitance (i.e.,

$${C}_{eq} = {C}_{1} + {C}_{2} = \frac{{Q}_{eq}}{V} = \frac{{Q}_{1}}{V} + \frac{{Q}_{2}}{V} ) .$$

Whereas, the reciprocal of the equivalent capacitance (C_{eq}) of two capacitors connected in series is the sum of the reciprocals of the individual capacitances (i.e.,

$$\frac{1}{{C}_{eq}} = \frac{1}{{C}_{1}} + \frac{1}{{C}_{2}} = \frac{{V}_{eq}}{Q} = \frac{{V}_{1}}{Q} + \frac{{V}_{2}}{Q} ) .$$

One method of using the CVD to measure capacitance of capacitance sensor 6 is to: (1) drive secondary channel to VDD as digital output, (2) point the ADC to the secondary VDD pin (charges C_{HOLD} to VDD), (3) ground the line of capacitance sensor 6, (4) turn the line of capacitance sensor 6 to input, (5) point the ADC to the channel of capacitance sensor 6 (voltage divider from capacitance sensor 6 to C_{HOLD}), (6) begin DC conversion, (7) read the ADC module register.

The basic principle begins with one ADC channel charging the internal sample-and-hold capacitor for the ADC to VDD. The channel of capacitance sensor 6 is then prepared to a known state by grounding it. After capacitance sensor 6 is grounded, it must be made an input again. Finally, immediately after it is made an input, the ADC channel is switched to the capacitance sensor 6. This puts the sample-and-hold capacitor, C_{HOLD}, in parallel with the capacitance sensor 6, creating a voltage divider between the two. Thus, the voltage on the capacitance sensor 6 is the same on the sample-and-hold capacitor. After this step, the ADC should be sampled, and the reading represents an amount of capacitance on capacitance sensor 6.

An attached microcontroller or connected computer measures the changes in the capacitance of capacitance sensor 6 and processes the changes to track volume level. The CVD method offers high immunity to noise as well as very low emissions. Sensing uses two ADC channels, but they may both be sensors. While one channel is actively scanning, the other sensor may be reused for a secondary line while scanning the first channel. While sensors are not being scanned, they should be kept at ground or VDD.

Bridge Method

Measuring capacitance of capacitance sensor 6 using a bridge approach or method involves the use of an AC Bridge for measuring capacitance. For example, FIG. 15 shows an unbalanced AC driven topology. The amount of unbalance is measured and is proportional to the capacitance of the capacitance sensor 6. As discussed above, as the liquid level in container 4 increases, the capacitance of capacitance sensor 6 changes. Accordingly, the unbalance, which is proportional to the capacitance of capacitance sensor 6, can be measured by a microcontroller or computer and processed to track volume level.

Charge-Based Method

Measuring capacitance of capacitance sensor 6 using a charge-based approach or method relies upon the ability of a capacitance sensor 6 to hold and transfer an electrical charge. The voltage present across a capacitor is proportional to the charge held in the capacitor (i.e.,

$$V = \frac{Q}{C} ) .$$

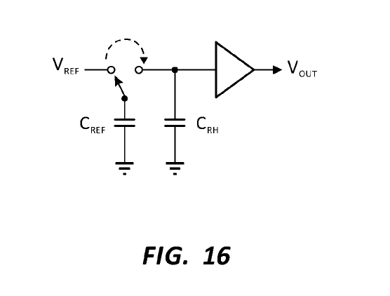

As depicted in FIG. 16, one method of measuring using this approach relies upon a reference Capacitor (C_{REF}) being charged by a known Voltage Source (V_{REF}) similar to the CVD capacitance voltage divider method discussed above. Depending on how the reference capacitor and capacitance sensor 6 are connected (in series or in parallel), one can solve for the capacitance of capacitance sensor 6 based on the information known about the reference capacitor and the measured data. An attached microcontroller or connected computer monitors changes in the capacitance of capacitance sensor 6 and processes the changes to track volume level.

Capacitive Sensing Module Method

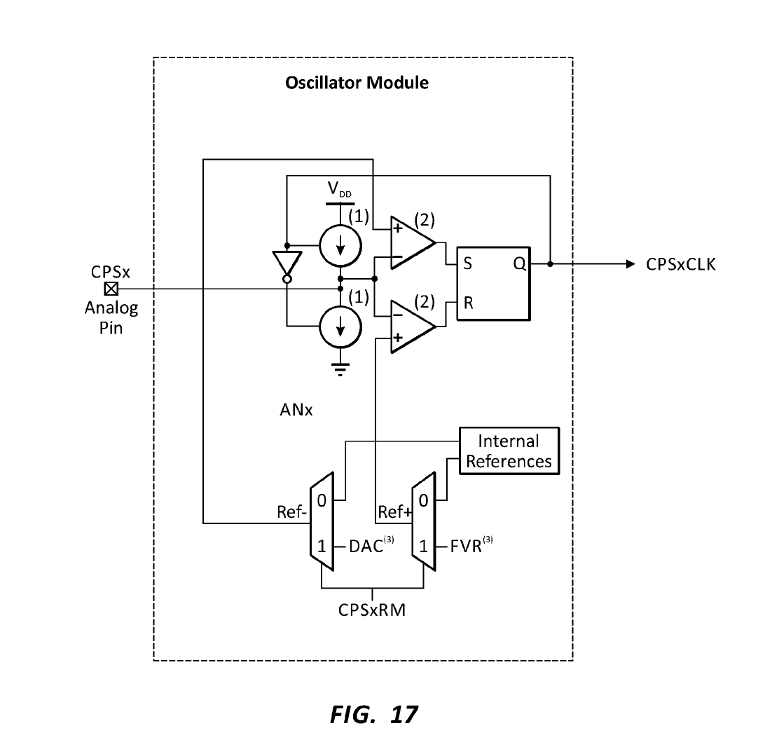

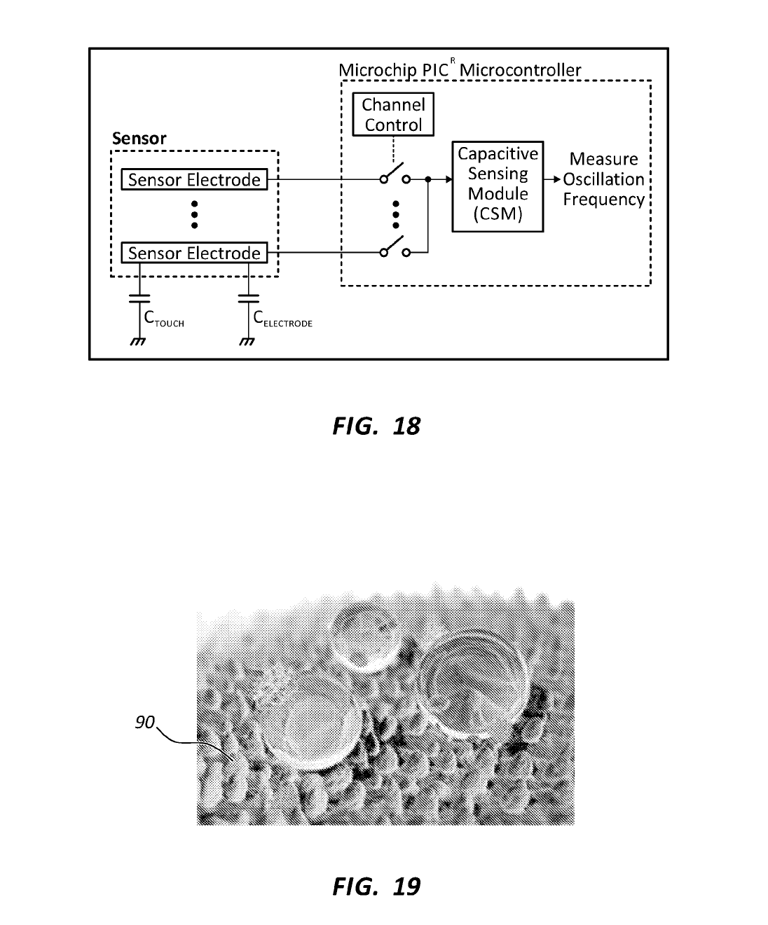

Capacitance may also be measured using a Capacitive Sensing Module (CSM) approach. FIG. 17 shows an example of a microchip microcontroller internal capacitive sensing module, and FIG. 18 shows an example of a CSM block diagram. A CSM approach simplifies the amount of hardware and software setup needed for capacitive sensing applications. Only the sensing electrodes on the collection bag need to be added. The capacitive sensing modules allow for an interaction with an end user without a mechanical interface. In a typical application, the capacitive sensing module is attached to an electrode of the urine collection bag, which is electrically isolated from the end user. When the urine enters the collection bag and starts displacing the air inside the bag, a capacitive load is added, causing a frequency shift in the capacitive sensing module.

The capacitive sensing module uses software and at least one timer resource (e.g., timer resources common on most microcontrollers) to determine the change in frequency. The change in frequency (as influenced by the change in capacitance) is measured by a microcontroller or computer and processed to track volume level. Some features of this module may include: analog multiplexer (MUX) for monitoring multiple inputs, a capacitive sensing oscillator, multiple Power modes, high power range with variable voltage references, multiple timer resources, software control, operation during sleep, and acquire two samples simultaneously (when using both CSM modules).

The CSM module capacitive sensing oscillator consists of a constant current source and a constant current sink, to produce a triangle waveform. The oscillator is designed to drive a capacitive load (single electrode) and at the same time, be a clock source to one of the timers. It has three different current settings as defined by appropriate registers. The different current settings for the oscillator serve at least two purposes: (1) to maximize the number of counts in a timer for a fixed time base; and (2) to maximize the count differential in the timer during a change in frequency.

### Methods for Improving Quality of Capacitance Measurement

The quality of measurement of a capacitance sensor may be affected by various factors, including system level variance and interference due to temperature, humidity, electrostatic discharge (ESD) and other stimuli. Various methods and means may be used to account for these factors and improve the quality of results. These methods, which are described in more detail below, can be used with any of the capacitance-based meters described herein.

For example, the dielectric constant of some materials varies with temperature which can affect the capacitance measured. To compensate, a temperature sensor or thermometer may be incorporated into the container 4 or associated equipment to monitor the temperature of the fluid. The capacitance sensor 6 can be calibrated at various temperatures and dielectric constant values to quantify the effect of any temperature changes. However, temperature compensation is not necessarily required in smart urine meter 2, e.g., urine may remain or be assumed to remain at approximately the average body temperature during urine collection and measurement.

Variations in composition of the measured liquid over time may also lead to some measurement error. Mixing materials with different dielectric constants in varying ratios can change the overall dielectric constant and the resultant capacitance generated. To compensate, two additional capacitors may be used, one that will be exposed to the fluid (e.g., compensation capacitor 10) and another that will be exposed to air (e.g., reference capacitor 8). This way any dielectric changes can be detected and compensated for in real time as discussed in more detail above.

Large variations in the conductivity of the measured material over time may also lead to some measurement error. However, proper electrode selection can minimize the effect. Thick wall electrode insulation is also recommended. Additionally, the use of a pair of capacitors to determine in real time the dielectric of the solution to be measured (e.g., as discussed above) can also help compensate for these variations.

Interfering electromagnetic signals can deteriorate the accuracy and the resolution of the measurement system. Indeed, the measurement of very small capacitances requires the use of very sensitive electronic circuits. Accordingly, the prevention of electromagnetic interference (EMI) plays an important role. Electromagnetic shielding can be used to eliminate or significantly reduce the impact of interfering electromagnetic signals. Electromagnetic shielding is the process of stopping the movement of an electric field in space. When an electric field is moving through space, and it hits an electric shield, it does two things: deflects most of it, and then the rest is observed by the actual shielding. The only electric energy that goes through is residual.

Many techniques can be applied to reduce electromagnetic interference such as the use of: (1) shielded boxes around the measurement circuits; (2) shielded cables; (3) (shielded) twisted pair cables; and (4) net filters.

Additional electromagnetic interference may be filtered out by the measurement system itself. Such filtering is possible when the frequency of the interference is substantially higher or lower than the frequency of operation of the measurement system. For example, most low-cost, capacitance measurement-based meters/systems will operate in the frequency range from 1 kHz to 1 MHz, so the interference caused by the electric mains (e.g., frequencies of 50 Hz (60 Hz in the US) and its harmonics (e.g., 65 Hz and high-frequency interference)), can be divided by frequencies well above 1 MHz caused by switching in digital circuits and by radio transmitters etc.

Parasitic capacitance (Cp) or additional capacitance caused by external noise can also create instability and reduce sensitivity in capacitive systems. Conducted noise and radiated noise are the most common types of interference noise. Conducted noise is caused in systems that are powered externally from the device. This can include systems powered off the main-line power, desktop-powered USB devices, or any other situation that may mean the user is not sharing a ground with the device. Radiated noise comes from electronic devices (e.g., cell phones) radiating electro-magnetic fields near the capacitive system.

The impact of parasitic capacitance can be reduced by amplifying the original value of the capacitance of the capacitor to make it greater than the parasitic capacitance. For example, the area of the electrodes can be designed to be much larger than the separation distance between electrodes, so that the relative impact from parasitic capacitance becomes negligible. The impact of Cp can also be reduced by using thin plates to decrease fringing fields on the margin of the electric field. This helps reduce the impact of Cp because capacitance is related with the shape of the electric field margin, which is closely related to the structure of the capacitance.

The impact of Cp can also be reduced by using appropriate electromagnetic shielding and grounding. Appropriate shielding and grounding not only decreases the surrounding interference (e.g., electromagnetic interference) but can also minimize the impact of the parasitic capacitance Cp. Additionally, the impact of Cp can be reduced by minimizing the length of the leader cable, i.e. make the presence of the circuit close enough to the capacitive sensor to decrease the impact of Cp.

Optionally, a small capacitor or a feedback circuit can also be used to generate a negative capacitance to cancel or reduce the effects of the parasitic capacitance. A positive feedback circuit provides the current lost through capacitance between the connecting points, preventing a potential drop across the electrode resistance. Good compensation will depend on the agility with which the feedback circuit can supply current. The fully compensated rise time is proportional to the geometric mean of the rise time of the recording amplifier and the rise time of the uncompensated circuit. One may also use shielding and/or other methods to minimize stray capacitance in combination with a head-stage amplifier with a fast rise time.

Additionally, if container 4 is a flexible bag/container, some electric-field bending around the bag may occur due to the natural bending of the bag as it is filled. This electric-field bending around the curvature of the bag can cause non-linearity in the results around the curvature. However, if the natural bending of the bag occurs in a predictable and consistent way, the urine meter 2 may be programmed to compensate or account for the electric-field bending of the bag. Alternatively, the electrodes of capacitance sensor 6 may be mounted on a more rigid or semi-rigid surface (e.g., one that is an integral part of the bag, or one that is attached to the bag) to inhibit bending of the electrodes and minimize the electric-filed bending effect.

Also, capacitors can leak current, which can create instability. Accordingly, it is preferable to construct urine meter 2 with capacitance sensor 6 between the ground pin and Earth potential. This arrangement solves problems with leakage current, which are more pronounced in floating capacitors. In this setup a galvanic isolation is established between the chosen sensor and Earth potential.

Additionally, surface tension of the tubing/catheter material (e.g., silicone) and/or the flow meter (e.g., flow meter 52) can cause the fluid passing therethrough to columnate instead of flowing continuously. Columnation can lead to the fluid (e.g., urine) backing up and not flowing properly though the tubing/catheter and/or other equipment. When columnation occurs it can be difficult to get an accurate measurement of flow rate. For example, the initial flow of urine can be delayed by the columnation and thereby prevent accurate measurement of initial flow. Additionally, when this columnation occurs, it can cause a bolus amount of fluid to form in the tubing/catheter. When the surface tension is overcome, the bolus amount of fluid is released, but the bolus can cause error in flow rate measurements. Another drawback, is that columniation can leave residual fluid “backed-up” in bladder and leave residual fluid in the drain lumen, which can lead to sanitation and health issues as well as errors in measurements.

To prevent columnation, a lubricious hydrophobic coating may be added to the inner surface of the lumen (e.g., drainage lumen) of any catheter/tubing used with the fluid measurement system. A similar lubricious hydrophobic coating may also be added to the surfaces of fluid passage 64 in flow meter 52.

Optionally, a surfactant solution can also be prepared and flushed through the drainage lumen, associated tubing, surfaces of fluid passage 64 in flow meter 52, and/or any other fluid passage surfaces. A surfactant may be added during manufacture or just prior to use to prevent columnation and ensure continuous flow. Optionally, a surfactant could be embedded in the wall/surfaces of the lumen/fluid passages, e.g., by mixing the surfactant into a dipping solution used to create the inner lumen layer of a catheter/tubing during a dipping manufacturing process. The external/outer surface of the catheter/tubing is generally not treated with the surfactant solution to preserve the characteristics of coatings already existing on the outer surface. For example, if the catheter/tubing already includes a polyurethane coating with antimicrobial silver oxide, the surfactant solution might interfere with the beneficial properties of the outer coating. Surfactant solutions that may beneficially be used to treat the lumens and fluid passages comprise fluorosurfactants, hydrocarbon surfactants, silicone surfactants, PFOS, Masurf FS-100, Masurf FS-115/FS-130, Masurf FS-130A, Masurf FS-130EB, Maurf FS-1400, Masurf FS-1700, Masurf FS-1725EB, Masurf FS-17401, Masurf FS-1750EG, Masurf FS-230, Masurf FS-2620, Masurf FS-2800, Masurf FS-2950, Masurf FS-3020, Masurf FS-3330A, Masurf FS-630, Masurf FS-710, Masurf FS-780, Masurf FS-810, Masurf FS-910, Masurf LA-130A, Masurf NF-10, Masurf NF-25, Masurf NRW, Masurf SP-1020, Masurf SP-320, Masurf SP-430, Masurf SP-430R, Masurf SP-535, Masurf SP-535A, Masurf SP-740, Masurf SP-820, Masurf SP-925, Masurf UV-150, Masurf FS-3240, Zonyl FS-300, Masurf FS-3130, Zonyl FS-510, Zonyl FS-610, Zonyl FSO, Zonyl FSE, Zonyl FSG, Zonyl FTS, Zonyl 9361, Zonyl FSO-100, Zonyl 8857A, Zonyl 8867L, FC-4430, FC-4432, FC5120, Flexipel S-11WS, Flexiwet AB-28, Flexiwet DST, Flexiwet NF, Flexiwet NF-80, Flexiwet NI-M, Flexiwet NI-M100, Flexiwet PD-100, Flexiwet PD-15, Flexiwet PD-30EB, Flexiwet Q-22, Flexiwet RFS-20A, Flexiwet SSE, Thetawet FS-8000, Thetawet FS-8020DB, Thetawet FS-8020EB, Thetawet FS-8050, Thetawet FS-8100, Thetawet FS-8150, Thetawet FS-8200, Thetawet FS-8250, Surfynol TG, EnviroGem 2010, Surfynol 104, Surfynol 1045, Surfynol 440, Surfynol 485, Carbowet 100, Carbowet 106, Carbowet 109, Carbowet 125, Carbowet 13-40, Carbowet 144, Carbowet 300, Carbowet 76, Carbowet DC11, etc. The surfactant selected should be one that is compatible with any lubricious coating already used on the inner lumen surface and, when used in a rinse solution, one that is an effective additive in the rinse solution to reduce surface tension and friction force on the inner lumen surface.

Additionally, a superhydrophobic patterned design 90 (see e.g., FIGS. 19 & 20) can be formed on the inner surface of the lumen (e.g., drainage lumen) of any catheter/tubing used with the fluid measurement system. A similar superhydrophobic patterned design 90 may also be formed on the surfaces of fluid passage 64 in flow meter 52. The patterned design 90 can be used to create superhydrophobic inner lumen surfaces and prevent columnation. The contact angles of a water droplet on a superhydrophobic surface may exceed 150° and the roll-off angle may be less than 10° making the superhydrophobic surface extremely difficult to wet.

Superhydrophobicity can be obtained by artificially adding small-scale roughness to hydrophobic surfaces to keep droplets in a Cassie Baxter state, i.e., a state in which air remains trapped inside the microscopic crevasses below the droplet. The roughness of a hydrophobic surface further decreases the wettability of the hydrophobic surface resulting in an increased water-repellency or superhydrophobicity. Wettability characteristics are those surface parameters which are directly linked to the wetting nature of materials; for instance, the contact angle is the angle the liquid droplet makes with the solid surface, and the surface free energy is the energy associated with the solid surface giving rise to the contact angle. Energetically the best configuration for the drop is on top of the corrugation like “a fakir on a bed of nails.” FIG. 19, shows droplets sitting on top of a rough superhydrophobic patterned surface 90.

Also, a droplet on an inclined superhydrophobic surface does not slide off; it rolls off. A benefit of this is that when the droplet rolls over a contamination, (e.g., dirt, dust, pollution, or viral/bacterial material, etc.) the contamination is removed from the surface if the force of absorption of the particle is higher than the static friction force between the particle and the surface. Usually the force needed to remove a particle/contamination is very low due to the minimized contact area between the particle/contamination and the surface. Accordingly, superhydrophobic surfaces have very good self-cleaning properties, and the growth of bacterial colonies is inhibited on the water-repellant surfaces.

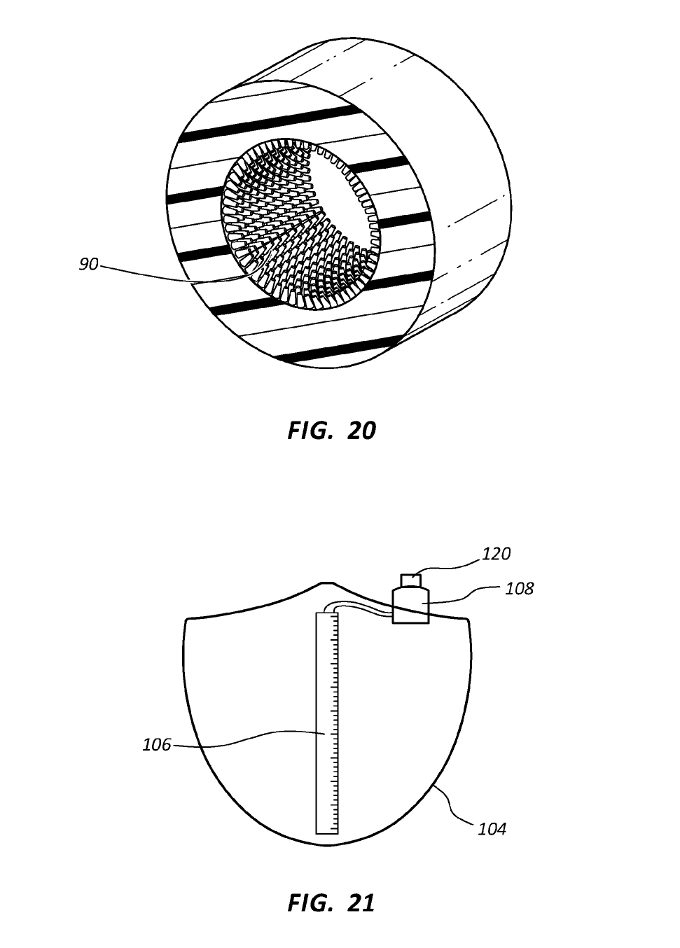

A superhydrophobic patterned surface 90, e.g., as shown in FIG. 20, may be formed on the inner surface of any tubing/catheter used in the system and on the inner surface of fluid passage 64 of flow meter 52 such that liquid droplets will always be in the Cassie Baxter state, which improves the drainage and fluid flow inside the tubing/catheter and flow meter 52. Preferably, the superhydrophobic patterned surface 90 has a liquid/urine contact angle greater than 150° for extraordinary liquid/urine repelling properties and to eliminate the fluid columnating inside the tubing/catheter and/or flow meter. Superhydrophobic patterned surface 90 may include tapered, cylindrical or squared microstructures (e.g., pillars) of a certain height and diameter and with a fixed pitch.

Superhydrophobic patterned surface 90 can be added to the inner surface of the tubing/catheter/flow meter by etching an inverse of the pattern into the outer surface of a dipping form or mold used to create the inner surface of the tubing/catheter and/or flow meter 52. Alternatively, one may attach an external flexible structure with an inverse of the pattern to the dipping form or mold. The inverse-patterned dipping form or mold may then be used in a dipping/molding manufacturing process to make the tubing/catheter or housing of flow meter 52.

Superhydrophobic surfaces can be fabricated from micro-arrays of RTV or any other type of polymer with pillars or posts pitches ranging from 450 to 700 microns. Preferably, the height of uniform pillars or post of a superhydrophobic surface is between 250 μm-500 μm, but the height can range as high as 800 μm. Optionally, UV cured silicone posts at 400 μm pitch fabricated by dispensing layers of adhesive on top of a flexible substrate can be used. In some embodiments, the posts or pillars have a diameter of between 50-175 μm. FIG. 20 shows an exemplary patterned microstructure formed on one portion of an inner drainage lumen (not to scale). Although FIG. 20 shows the exemplary superhydrophobic patterned surface 90 as being on only one portion of the lumen surface, it is contemplated that the entire surface of the lumen will include the superhydrophobic patterned surface 90.

One method of forming the microstructures (e.g., pillars or posts) of superhydrophobic patterned surface 90 is using a laser to form the patterned microstructure directly on the desired surface, or using a laser to form the inverse of the pattern on the surface of a dipping form or mold that is then used to create the desired surface. The dipping form can then be dipped coated with a polymeric material to form a catheter or other tubing with the desired microstructure patterned surface. Lasers can be used on the surfaces of many different materials ranging from ceramics, to metals, to polymers. Lasers have the ability to change both the surface dimensions (roughness and surface pattern) and the surface chemistry simultaneously which can then lead to a change in the wettability characteristics.

Superhydrophobic patterned surfaces can also be prepared with a wide variety of surface shapes using a commercially available 3D printer. Fabrication of large, complex polymer objects on a flat surface that later can be incorporated into the form, for the dipping process. This can be achieved where the micro-textured surface is monolithic with the body or flexible structure. The superhydrophobic behavior, such as the water column height supported, can be described by the same equations as those used to describe superhydrophobic behavior on surfaces with nano-scale textural features.

Although discussed herein in the context of capacitance-based measurement systems, the superhydrophobic patterned surfaces would also be beneficial in Foley catheters and other tubing used with other types of flow meters, e.g., the additional measurement systems discussed below. Indeed, the superhydrophobic patterned surfaces would also be beneficial in catheters, tubing, flow through devices, etc. even if not connected to a meter or if used in a different context. Further, although discussed herein in terms of tubing, catheters, and flow meters associated with urine drainage/collection, the superhydrophobic patterned surfaces may be added to other types of medical tubing, catheters, and equipment through which fluid flows, e.g., dialysis catheters and equipment, vascular catheters, etc.

### Additional Measurement Systems

Various additional high resolution, low cost fluid monitoring systems are also contemplated. In general, a sensor or multiple sensors may be integrated with a fluid collection container/bag to form a smart urine meter or monitoring system that can sense volume, flow rate, and other parameters. The sensor(s) may respond to a physical stimulus (such as weight, heat, light, sound, pressure, magnetism, or a particular motion) and transmit a resulting impulse (as for measurement or operating a control). The performance of the sensor(s) can be considered in terms of physical units; i.e., kgf, mL, etc. The sensor or sensors may be integrally built into the container. Analog measurements from the sensors can be converted to digital in an analog to digital converter (ADC) and processed using a programmed microcontroller.

FIG. 21 shows a reliable, low cost fluid monitoring device or system in the form of a urine meter or monitoring system 102. Although described in terms of a urine monitoring system, the devices, systems, and principles described may be used in other fluid monitoring applications not related to urine collection and monitoring. As shown in FIG. 21, urine meter 102 may include a fluid collection container 104, a sensor 106, a microcontroller 108, and a wireless transceiver 120.

A wide variety of types of fluid collection containers or bags may be used for fluid collection container 104. Indeed, container 104 may be the same as or similar to any of the fluid collection containers or bags discussed above with respect to container 4 of urine meter 2, and may include any of the same features, shapes, sizes, materials, designs, etc. as container 4. Container 104 may be flexible, rigid, semi-rigid, and/or a combination of these. In practice, the container 104 may be designed to fill with fluid from the top or the bottom of the container, e.g., urine can flow from a Foley catheter into tubing associated with the container that empties into the container 204. In one embodiment, fluid flows through tubing connected at the top of container 204 to fill the container 204.

Sensor 106 is a printed electronic resistive sensor, e.g., an E-Tape liquid level sensor. A printed electronic resistive sensor is a solid state sensor that makes use of printed electronics instead of moving mechanical parts. The printed electronic resistive sensor is compressed by hydrostatic pressure of the fluid in which it is immersed resulting in a change in resistance which corresponds to the distance from the top of the sensor to the fluid surface. The volume of the liquid (e.g., urine) can be measured by correlating the modality hydrostatic pressure to volume using the printed electronic resistive sensor. In operation, as the liquid or urine level rises in the container/bag, the measured resistance decreases. The higher the liquid level, the lower the resistance. (Conversely, if the liquid level were to decrease, the resistance would increase.)

Sensor 106 is preferably able to measure a sufficient range to simulate urine output, be precise in measurements, and provide repeatable results to within an error of at least +/−5 mL. More preferably, the sensor will be able to provide repeatable results to within an error of at least +/−2 mL. The printed electronic resistive sensor 106 may be included within and/or as an integral part of the collection container 104. Optionally, the printed electronic resistive sensor may be adhered or otherwise attached to one side of the bag, such that only the non-adhered side faces the liquid. A benefit of sensor 106 is that it works equally well regardless of the shape or flexibility of the container 4.

For a simple resistance-to-voltage conversion, the printed electronic resistive sensor 106 is tied to a measuring resistor in a voltage divider configuration. The output can be described by the equation below:

$$Vout = \frac{Vs}{1 + \frac{Rtape}{Rm}}$$

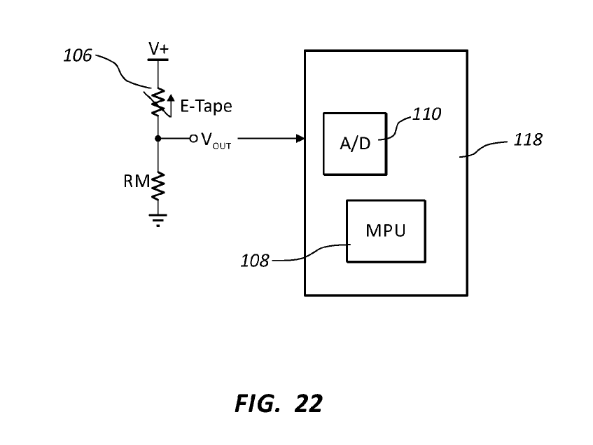

Microcontroller 108 may be attached to or otherwise integrated with container 4 or be part of an integrated circuit that is attached to container 4. FIG. 22 shows one example of a simplified sensor and wiring diagram for an embodiment employing an E-Tape liquid level sensor. In FIG. 22, microcontroller 108 is part of an integrated circuit 118, and is in communication with an analog to digital converter (ADC) 110. Analog voltage measurements from the sensor 106 are converted to digital in the ADC 110 and processed using the microcontroller 108.

The ADC 110 may be selected to meet resolution requirements of a particular application. For example, ADC 110 may be selected to have an output size preferably between 10 bits and 32 bits, which should meet most resolution requirements. However, higher output size ADCs may also be used. The bit value of the ADC corresponds directly to its resolution, and thereby refers to how finely it slices its full-scale measurement range, or in other words, the smallest change in the input signal that it can theoretically measure (ignoring noise). A higher bit value corresponds to better resolution.

The input resolution of an ADC used in the system can be calculated according to the following formula:

vd=System full Scale range
vs=Transducer Full Scale Range
E=Needed Full scale output
n=ADC number of bits
B=0 unipolar or B=1 for Bipolar

$$Resolution = ( \frac{vd}{vs * {2}^{n - B}} ) * E$$

For example, in an embodiment using a 12 bit ADC will give a maximum theoretical resolution of 0.54 g per bit. This can be calculated using a reference voltage of 3.3 Vdc, and a volume resolution assuming a correlation corresponding to 1 g=1 mL.

$$Res = ( \frac{3.3 V}{3.3 V * {2}^{12 - 0}} ) * 2200 g = 0.54 g$$

In the microcontroller 108, the measurement data from the sensor 106 and ADC 110 is processed by written software or firmware. This software/firmware consists of functions which combine the measurements data to produce usable quantities for the user. For example, as discussed below, the measurement signal from sensor 106 may be correlated to fluid volume by curve fitting the data (e.g., based on Lagrange interpolation). The relationship (e.g., curved or linear equation) between the measurement readings and a particular volume may be programmed into the software/firmware, so that volume may be calculated based on the sensor readings. The software/firmware may also be programmed to track the volume level over time to calculate flow rate. The software/firmware may also signal that the volume level, flow rate, and any other measured/calculated parameters be displayed on or transferred to a monitor, computer, smart phone, and/or other device. The parameters may be continuously calculated, updated, and displayed in real time, e.g., during urine collection. The software/firmware may also be programmed to accomplish other purposes/functions, including those discussed elsewhere herein.

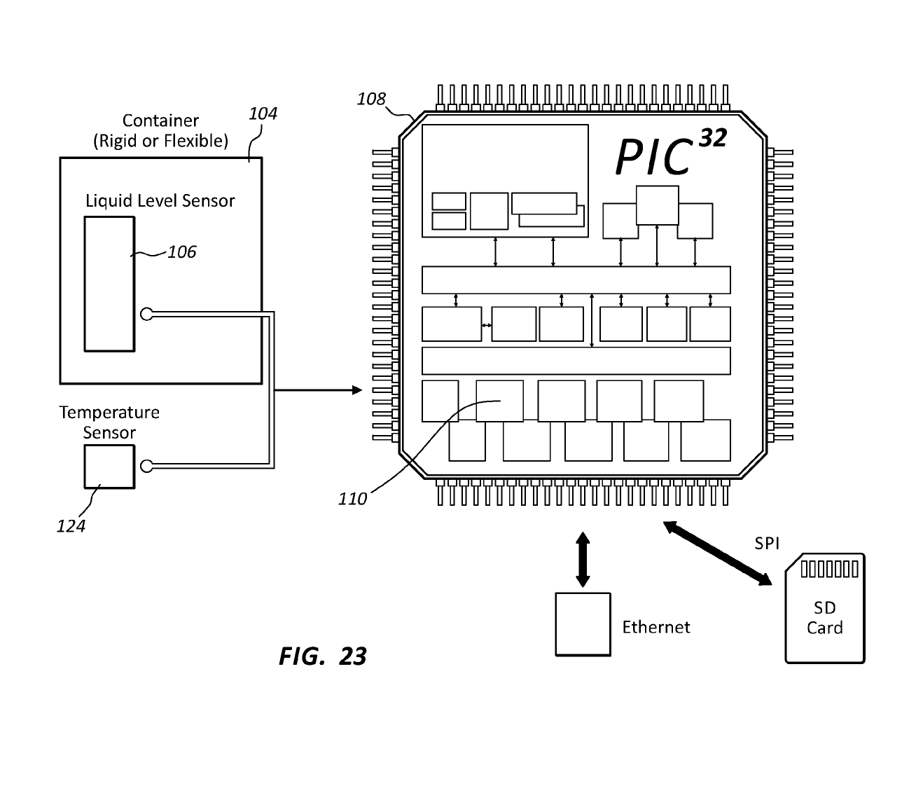

In one embodiment, as shown in FIG. 23, microcontroller 108 is a 32-bit PIC 32 microcontroller. The PIC32 board provides a complete, high-quality development platform for PIC32MX7 series devices. It has numerous on-board modules (Ethernet PHY), I2C, SPI, RTC, audio codec, accelerometer, temperature sensor, and flash memory, which allows to write applications of high complexity quicker. In this and other embodiments, the ADC 110 is built in to or integrated with the microcontroller 108. As shown in FIG. 23, a temperature sensor 124 may also be used to feed temperature data to the microcontroller 108. The data from temperature sensor 124 may be processed and displayed with measurement/calculated data from other sensors, e.g., sensor 106. Temperature sensor 124 may optionally be integrated into urine meter 102, e.g., built in or attached to container 104.

Various other microcontrollers can optionally be used in the system. For example, other boards with similar modules and functions may be used, e.g., higher bit boards or boards with additional modules. Additionally, microcontrollers and/or integrated circuits disclosed above with respect to urine meter 2 may also be used.

For software calibration and curve-fitting/linearization of the measured data, a weight scale may be initially used to correlate the reported volume of the liquid with an actual experimental volume poured. For the hardware/software to calculate a value for volume based on the output data measured from sensor 106, a relationship between the output voltage of the circuitry and the volume (which may be estimated by the applied weight using a weight scale as mentioned above) is first determined. One way to do this is to use Excel and/or Minitab software to calculate an nth degree polynomial curve relating the output voltage of the circuitry and the applied weight or volume in order to interpolate the applied weight or volume corresponding to voltage outputs. Thereby an equation (e.g., a predictable curve or line equation) can be found that describes the sensor behavior.

Alternatively, the relationship between the output voltage of the circuitry and the applied weight or volume may be determined using a Lagrange interpolation method in real time using an appropriate algorithm. For example, the following real-time Lagrange curve fit algorithm may be used.

```
Function Lagr2 (periodcol As Range, ratecol As Range, X)
Dim V0, V1, V2, V3, V, Vol0, Vol1, Vol2, Vol3, Vol, L0, L1, L2, L3,
FindVolt As Double
Dim i As Integer
period_count = periodcol.Rows.Count
ReDim Voltage(period_count) As Single
ReDim Volume(period_count) As Single
For c = 1 To period_count
  Voltage(c) = periodcol(c)
  Volume(c) = ratecol(c)
Next c
FindVolt = X
```

For i = 1 To period_count

If (FindVolt <= Voltage(i)) Then

If i > 2 Then

V0 = Voltage(i − 2)
V1 = Voltage(i − 1)
V = Voltage(i)
V2 = Voltage(i + 1)
V3 = Voltage(i + 2)
Vo10 = Volume(i − 2)
Vo11 = Volume(i − 1)
′Vol = Volume(i) //unknown??
Vo12 = Volume(i + 1)
Vo13 = Volume(i + 2)

End If

End If

Next i
′Lagrange Interpolation
L0 = ((V − V1) / (V0 − V1)) * ((V − V2) / (V0 − V2)) * ((V − V3) /
(V0 − V3))
L1 = ((V − V0) / (V1 − V0)) * ((V − V2) / (V1 − V2)) * ((V − V3) /
(V1 − V3))
L2 = ((V − V0) / (V2 − V0)) * ((V − V1) / (V2 − V1)) * ((V − V3) /
(V2 − V3))
L3 = ((V − V0) / (V3 − V0)) * ((V − V1) / (V3 − V1)) * ((V − V2) /
(V3 − V2))
Vol = (L0 * Vo10) + (L1 * Vol1) + (L2 * Vol2) + (L3 * Vol3)
Lagr2 = Vol

End Function

This real-time Lagrange curve fit algorithm was tested using data from a prototype urine meter using an E-tape electronic resistive sensor, and worked well to provide reasonably accurate data. For example, Table 1 below shows minimal error between the experimental and calculated Lagrange volume.

TABLE 1

| | Experimental | | | |
|---|---|---|---|---|
| Voltage | Volume | Lagrange Volume | Error | |
| 2.9 | 167.3 | 167.6333313 | −0.33333 | Over |
| 2.896774 | 181.1 | 182.3000183 | −1.20002 | Over |
| 2.893548 | 197.2 | 194.9500122 | 2.249988 | Under |
| 2.890323 | 207 | 207.3666687 | −0.36667 | Over |
| 2.887097 | 215.4 | 216.6000061 | −1.20001 | Over |
| 2.883871 | 225.1 | 224.2666626 | 0.833337 | Under |
| 2.880645 | 231.6 | 230.066864 | 1.533136 | Under |

Urine meter 102 may optionally include a wireless transceiver 120. Wireless transceiver 120 may be the same as or similar to the wireless transceivers discussed with respect to urine meter 2 (e.g., Zigbee, etc.), to wirelessly communicate with a remote computer or unit to improve the usability of the system. Alternatively, urine meter 102 may include one of the other means of communication disclosed above with respect to urine meter 2. With respect to data transmission, urine meter 102 can function in the same way as urine meter 2, as discussed above.

Further, multiple urine meters each connected to a different patient can also be configured to transmit data to the same computer or network as discussed above with respect to urine meter 2. Software associated with each urine meter can be programmed to transmit the measured data with a unique identifier to distinguish the data transmitted by one urine meter from the data transmitted by each of the other urine meters.

The urine meter 102 may also include a display or monitor that is programmed to display volume, flow rate, temperature, and/or other parameters based on sensor measurements.

Printed electronic resistive sensors tend to have a “blind inch” or so where the sensor does not sense the water level, i.e., because the water pressure is not yet high enough to register on the sensor. To compensate for this a pressure sleeve or similar device may be attached to the base of the printed electronic resistive sensor to provide a certain amount of initial pressure on the sensor to help overcome the “blind inch.” Alternatively, the volume corresponding to the “blind inch” may have a known value that is automatically added to the volume by the microprocessor once the sensor begins sensing (i.e., the water level exceeds the “blind inch”). Optionally, the printed electronic resistive sensor may be combined with another sensor disclosed herein, which may measure the volume and flow rate until the printed electronic resistive sensor begins sensing.

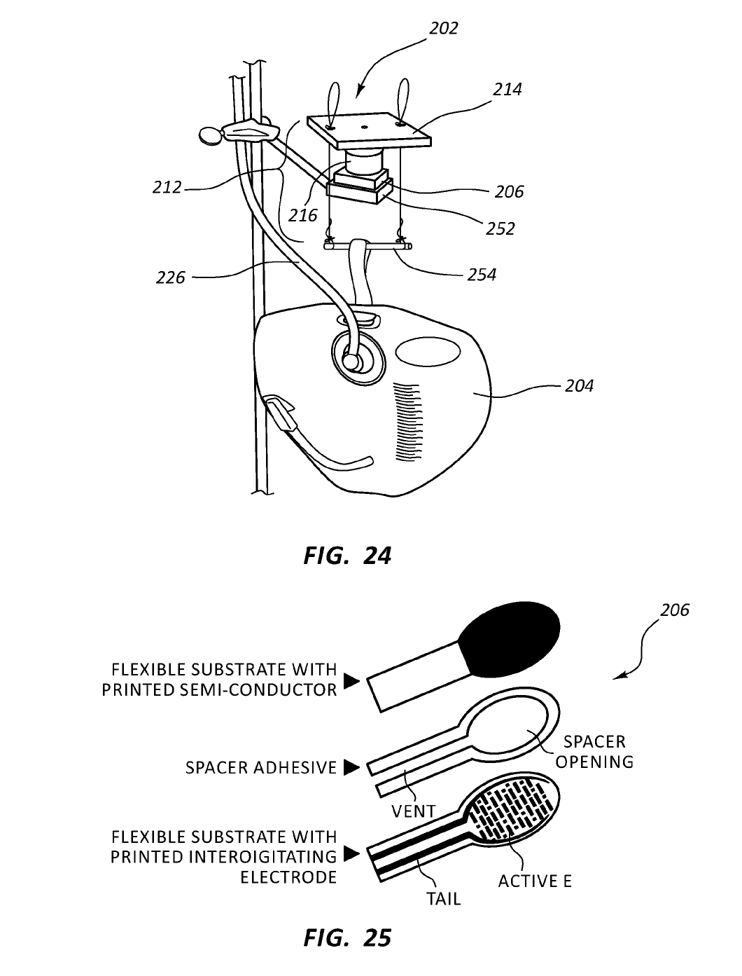

FIG. 24 shows a reliable, low cost fluid monitoring device or system in the form of a urine meter or monitoring system 202. Although described in terms of a urine monitoring system, the devices, systems, and principles described may be used in other fluid monitoring applications not related to urine collection and monitoring. As shown in FIG. 24, urine monitoring system 202 may include a fluid collection container 204 and a support and measurement assembly 212. Support and measurement assembly 212 may include a sensor 206, a contact object 216, and a supported lower platform 252. In one embodiment, support and measurement assembly 212 may also include a platform 214 and a cross beam 254 attached to platform 214 using wires or other connectors such that downward force on cross beam 254 is transferred to platform 214.

A wide variety of types of fluid collection containers or bags may be used for fluid collection container 204. Indeed, container 204 may be the same as or similar to any of the fluid collection containers or bags discussed above with respect to container 4 of urine meter 2, and may include any of the same features, shapes, sizes, materials, designs, etc. as container 4. Container 204 may be flexible, rigid, semi-rigid, and/or a combination of these. In practice, the container 204 may be designed to fill with fluid from the top or the bottom of the container, e.g., urine can flow from a Foley catheter into tubing associated with the container that empties into the container 204. In one embodiment, fluid flows through tubing 226 connected at the top of container 204 to fill the container 204.

Sensor 206 is a Force-Sensing Resistor (“FSR”). The FSR can be made of a polymer thick film ink, typically screen printed on Mylar film, depending on the requirements of the application; as force is applied to the device, the electrical resistance decreases. FSRs can be used to create an ultra-low cost urine meter for measuring volume and flow rate. Some of the components of an FSR are shown in FIG. 25. The ink formulation of an FSR can be customized for application-specific requirements, such as minimizing saturation with greater force, as well as for very low forces needs. Temperature, humidity, and shear are some of the considerations. The FSR used is preferably able to measure forces caused by applied weights up to at least 2.5 kg to simulate urine output, be precise in measurements to less than or equal to 3 kg, and provide repeatable results to within an error of at least +/−5 mL.

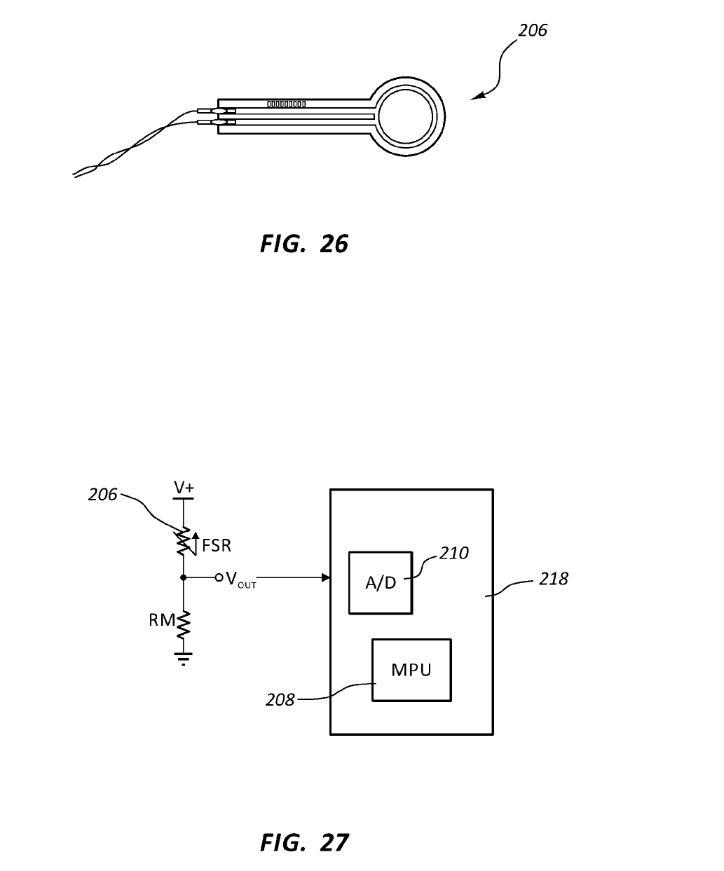

A design of a mechanical fixture for holding the sensor contact area constant and preventing bending and subsequently converting the FSR into a force sensor can be seen in FIG. 26. A solid structure that will uniformly distribute the pressure exerted on the sensor is generally needed, independent of the actuator contact area. Having the sensor contact area constant, the repeatability will be increased, and the FSR will act as good repeatable force sensor with good accuracy.

Additionally, covering the FSR with a rubbery or soft overlying layer will distribute the applied load effectively, increasing the slope of the force/voltage curve at low forces and a decreasing the slope at high forces.

FSRs have many advantages, including a small size, low weight, being inexpensive, and being easy and versatile to utilize. However, some difficulties can arise when the FSR is exposed to non-uniform pressures and mechanical moments. However, the set-up of the sensor 206, support and measurement assembly 212, and the methods of calibration can be refined to maximize reliability and accuracy. One such adjustment, in order to improve sensor repeatability, is to attach a solid structure or contact object 216 to the pressure sensing area holding the sensor contact area constant and preventing bending, and subsequently converting the FSR into a force sensor.

In one embodiment, the fluid container 204 may be arranged such that it hangs from a support and measurement assembly 212. FIG. 24 shows one potential arrangement for support and measurement assembly 212; however other arrangements designs in keeping with the principles described herein are also contemplated. In FIG. 24, support and measurement assembly 212 includes a platform 214 connected to a cross beam 254 (from which the fluid container 204 hangs) and a contact object 216 attached to the bottom of the platform 214, such that contact object 216 is disposed directly above the FSR in contact with the surface of the FSR (i.e., sensor 206) (if an overlying layer is used as discussed above, the overlying layer may be considered the surface of the FSR). As the fluid container 204 fills with fluid (e.g., urine), its weight increases and it pulls more heavily downward on platform 214, which pushes contact object 216 more strongly against the sensor 206. In this way, an FSR can be used with any size or type of fluid container to measure the increase in weight or downward force as the fluid container 4 fills with fluid. This downward force or weight increase can be correlated to volume increase to give a measurement of volume and flow rate. Other arrangements for using an FSR are also contemplated, for example, fluid container 4 may have an FSR disposed at or near the base of fluid container 4 arranged such that as the liquid/urine fills the container its weight is focused downward on the FSR.

The FSA sensor can be used to create a Voltage divider. For a simple force-to-voltage conversion, the FSR device is tied to a measuring resistor in a voltage divider configuration. The output equation could be described by the equation below:

$$Vout = \frac{Vs}{1 + \frac{RFsr}{Rm}}$$

A microcontroller, which may be the same or similar to microcontroller 108 discussed above, may be attached to or otherwise integrated with container 204 or be part of an integrated circuit that is attached to container 204. Alternatively, the microcontroller may be attached to or otherwise integrated with support and measurement assembly 212 or be part of an integrated circuit that is attached to support and measurement assembly 212. FIG. 27 shows one example of a simplified sensor and wiring diagram for an embodiment employing an FSR sensor. In FIG. 22, microcontroller 208 is part of an integrated circuit 218, and is in communication with an ADC 210. Analog voltage measurements from the sensor 206 are converted to digital in the ADC 210 and processed using the microcontroller 208. The ADC 210 may be the same as or similar to ADC 110 discussed above. The ADC 210 may be selected to meet resolution requirements of a particular application in the same way discussed above with respect to ADC 110.

In the microcontroller 208, the measurement data from the sensor 206 and ADC 210 is processed by written software or firmware similar to how this is done in microcontroller 108 (discussed above). This software/firmware consists of functions which combine the measurements data to produce usable quantities for the user, as discussed above. The measurement signal from sensor 206 may be correlated to fluid volume by curve fitting the data (e.g., based on Lagrange interpolation). The relationship between the measurement readings and a particular volume may be programmed into the software/firmware, so that volume, flow rate and/or other parameters may be calculated based on the sensor readings. The software/firmware may also signal that the volume level, flow rate, and any other measured/calculated parameters be displayed on or transferred to a monitor, computer, smart phone, and/or other device. The parameters may be continuously calculated, updated, and displayed in real time, e.g., during urine collection. The software/firmware may also be programmed to accomplish other purposes/functions, including those discussed elsewhere herein.

In one embodiment, microcontroller 208 is a 32-bit PIC 32 microcontroller similar to that shown in FIG. 23 and discussed above. In this and other embodiments, the ADC 210 is built in to or integrated with the microcontroller 208. A temperature sensor similar to temperature sensor 124 may also be used to feed temperature data to the microcontroller 208. The data from the temperature sensor may be processed and displayed with measurement/calculated data from other sensors, e.g., sensor 206. The temperature sensor may optionally be integrated into urine monitoring system 202, e.g., built in or attached to container 204.

Various other microcontrollers can optionally be used in the system. For example, other boards with similar modules and functions may be used, e.g., higher bit boards or boards with additional modules. Additionally, microcontrollers and/or integrated circuits disclosed above with respect to urine meter 2 may also be used.

As with printed electronic resistive sensor 106, linearization and/or curve fitting functions with the FSR can be accomplished using Minitab and/or excel or by using the above real-time Lagrange curve fit algorithm. However, the real-time Lagrange curve fit algorithm simplifies the calculations within the software and tends to give a better relationship between the output voltage of the circuitry and the applied weight. The behavior of each of these curves is dependent on the surface size of the sensor, the relative area on the sensor that is being utilized, and the fixed voltage value that is placed in the voltage divider circuit.

Optionally, conductance may be plotted vs. force (the inverse of resistance: 1/r). This format allows interpretation on a linear scale. For reference, the corresponding resistance values may also be included. A simple circuit called a current-to-voltage converter can give a voltage output directly proportional to FSR conductance and can be useful where response linearity is desired to avoid complex curve fitting. The FSR has a strong logarithmic relation for resistance versus pressure.

A test was carried out to study the capabilities of a prototype using an FSR sensor. As part of the test, the same real-time Lagrange curve fit algorithm as discussed above was used. As shown in Table 2, the error between the experimental and calculated Lagrange volume is fairly reasonable.

TABLE 2

| Voltage | Experimental Volume | Lagrange Volume | Error | |
|---|---|---|---|---|
| 0.237581 | 87.1 | 87.06278229 | 0.037218 | under |
| 0.245171 | 88.22 | 89.16555786 | −0.94556 | over |
| 0.249057 | 89.2 | 89.98143768 | −0.78144 | over |
| 0.253067 | 90.07 | 93.07049561 | −3.0005 | over |
| 0.284728 | 91.01 | 91.72317505 | −0.71318 | over |
| 0.315932 | 92.1 | 91.72317505 | 0.376825 | under |

If the FSR experiences issues with the resistance value drifting somewhat over time, these issues with drift can be prevented by calibrating FSR sensors at least once a week to ensure proper force measurements are being taken.

Urine monitoring system 202 may optionally include a wireless transceiver. The wireless transceiver may be the same as or similar to the wireless transceivers discussed with respect to urine meter 2 (e.g., Zigbee, etc.), to wirelessly communicate with a remote computer or unit to improve the usability of the system. Alternatively, urine monitoring system 202 may include one of the other means of communication disclosed above with respect to urine meter 2. With respect to data transmission, urine monitoring system 202 can function in the same way as urine meter 2, as discussed above.

Further, multiple monitoring systems each connected to a different patient can also be configured to transmit data to the same computer or network as discussed above with respect to urine meter 2. Software associated with each urine monitoring system can be programmed to transmit the measured data with a unique identifier to distinguish the data transmitted by one urine monitoring system from the data transmitted by each of the other urine monitoring systems.

The urine monitoring system 202 may also include a display or monitor that is programmed to display volume, flow rate, temperature, and/or other parameters based on sensor measurements.

The above fluid monitoring systems have generally been described as being applied to a urine meter(s) or urine monitoring systems; however, the principles described may be applied to other types of fluid measurement or monitoring systems, i.e., not involving urine. Further, the features described in one embodiment may generally be combined with features described in other embodiments. For example, the tilt sensing feature of the capacitance sensor may be combined with the printed electronic resistive sensor system. Also, the hydrophobic coating, surfactant treatment, and superhydrophobic patterned surface features may be included on tubing/catheters associated with any of the embodiments disclosed herein. Indeed, in some cases more than one type of monitoring system or sensor may be combined. For example, printed electronic resistive sensors tend to have a “blind” inch or so where the sensor does not sense the water level, i.e., because the water pressure is not yet high enough to register on the sensor. Accordingly, a capacitance-based sensor or FSR sensor may be used to sense the first inch, then the printed electronic resistive sensor can take over.

Components of the apparatuses, devices, systems, and methods described herein may be implemented in hardware, software, or a combination of both. Where components of the apparatuses, devices, systems and/or methods are implemented in software, the software (e.g., software including algorithms/calculations discussed above) may be stored in an executable format on one or more non-transitory machine-readable mediums. Further, the algorithms, calculations, and/or steps of the methods described above may be implemented in software as a set of data and instructions. A machine-readable medium includes any mechanism that provides (e.g., stores and/or transports) information in a form readable by a machine (e.g., a computer). For example, a machine-readable medium includes read only memory (ROM); random access memory (RAM); magnetic disk storage media; optical storage media; flash memory devices; DVD's, electrical, optical, acoustical or other form of propagated signals (e.g., carrier waves, infrared signals, digital signals, EPROMs, EEPROMs, FLASH, magnetic or optical cards, or any type of media suitable for storing electronic instructions. The information representing the apparatuses and/or methods stored on the machine-readable medium may be used in the process of creating the apparatuses, devices, systems, and/or methods described herein. Hardware used to implement the invention may include integrated circuits, microprocessors, FPGAs, digital signal controllers, and/or other components.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A fluid monitoring system, comprising:
a container for collecting a fluid; and
a capacitance sensor attached to the container and configured to act as a capacitor to sense a physical property of the fluid as it collects in the container.

2. The fluid monitoring system according to claim 1, further comprising a microcontroller programmed to calculate a volume measurement of the fluid as it collects in the container based on data received from the capacitance sensor.

3. The fluid monitoring system according to claim 1, wherein the capacitance sensor has a generally coplanar electrode structure formed from only two parallel electrodes.

4. The fluid monitoring system according to claim 1, wherein the capacitance sensor is has an interdigital electrode structure.

5. The fluid monitoring system according to claim 4, wherein the interdigital electrode structure is formed from conductive ink on an external surface of the container.

6. The fluid monitoring system according to claim 2, further comprising a reference capacitor configured to measure a dielectric property of air and a compensation capacitor configured to measure a dielectric property of the fluid, the microcontroller programmed to continuously estimate a dielectric constant of the fluid based on data received from the reference capacitor and the compensation capacitor.

7. The fluid monitoring system according to claim 2, further comprising a wireless transceiver for transmitting the volume measurement to a separate device.

8. The fluid monitoring system according to claim 7, wherein the microcontroller includes software programmed to transmit the volume measurement with a unique identifier to distinguish the volume transmitted by the fluid monitoring system from data transmitted by other monitoring systems.

9. The fluid monitoring system according to claim 1, further comprising tubing through which the fluid flows before collecting in the container, an inner surface of the tubing including a surfactant.

10. The fluid monitoring system according to claim 9, wherein the surfactant is embedded in the inner surface.

11. A method of measuring fluid volume, comprising:
providing a urine monitoring device, comprising:
a container for collecting a fluid,
a capacitance sensor attached to the container and configured to act as a capacitor to sense a physical property of the fluid, and
a microcontroller programmed to use data from the capacitance sensor to calculate a volume of the fluid as it collects in the container,
calculating a volume of the fluid as it collects in the container based on data from the capacitance sensor.

12. The method according to claim 11, further comprising calculating a base capacitance of the capacitance sensor prior to calculating a volume of the fluid.

13. The method according to claim 11, wherein the data from the capacitance sensor is representative of a capacitance of the capacitance sensor, and the calculating a volume further comprises calculating a volume based on the data representative of the capacitance of the capacitance sensor.

14. The method according to claim 13, wherein the data representative of a capacitance of the capacitance sensor is measured indirectly from the changing frequency of an oscillator.

15. The method according to claim 11, further comprising calculating a flow rate of the fluid as it collects in the container based on data from the capacitance sensor.

16. A flow meter, comprising:
a housing including a fluid passage therethrough; and
a capacitance sensor inside the housing configured to act as a capacitor to sense a physical property of the fluid as it passes through the fluid passage.

17. The flow meter according to claim 16, further comprising a microcontroller programmed to calculate a volume of the fluid as it passes through the fluid passage based on data received from the capacitance sensor.

18. The flow meter according to claim 16, wherein the capacitance sensor has a coaxial electrode structure disposed around the fluid passage.

19. The flow meter according to claim 16, wherein the capacitance sensor has an electrode structure including two semicircular plates, the fluid passage disposed between the two semicircular plates.

20. The flow meter according to claim 16, further comprising a wireless transceiver for transmitting data to a separate device.

21. The flow meter according to claim 16, wherein an inner surface of the fluid passage includes a surfactant.

22. The flow meter according to claim 21, wherein the surfactant is embedded in the inner surface.

23. A urine monitoring system, comprising:
a container for collecting urine,
a printed electronic resistive sensor attached to an internal surface of the container and configured to measure a physical property of the urine as it collects in the container, and
a microcontroller programmed to calculate a volume of the urine as it collects in the container based on data received from the printed electronic resistive sensor.

24. A urine monitoring system, comprising:
a container for collecting urine,
a force-sensing resistor configured to provide a measurement value indicative of volume of the urine as it collects in the container,
a support and measurement assembly from which the container hangs, the support and measurement assembly including a contact object disposed directly above and in contact with the force-sensing resistor;
a microcontroller programmed to calculate a volume of the urine as it collects in the container based on the measurement value from the force-sensing resistor.
